(19) United States
(12) Patent Application Publication
(10) Pub. No.: US 2012/0083776 A1
(43) Pub. Date: Apr. 5, 2012

(54) ANNULAR AND OTHER ABLATION PROFILES FOR REFRACTIVE SURGERY SYSTEMS AND METHODS

(75) Inventors: Guang-ming Dai, Fremont, CA (US); Michael Brownell, San Clemente, CA (US)

(73) Assignee: AMO Development, LLC, Santa Ana, CA (US)

(21) Appl. No.: 12/897,946

(22) Filed: Oct. 5, 2010

(51) Int. Cl. A61F 9/008

(52) U.S. Cl. 606/5

(57) ABSTRACT

Systems, methods, and computer program products are provided for the administration of annular and other ablation profiles during refractive surgery treatments. Basis data framework techniques enable the implementation of both circular and annular ablation profiles resulting in increased ablation efficiency when treating certain vision conditions. In some instances, systems or treatments involve the use of symmetric and asymmetric ablations shapes such as double spots, triple spots, quadruple spots, multiple spots, arc shapes, elliptical shapes, and the like.

### CROSS-REFERENCES TO RELATED APPLICATIONS

This application is related to U.S. Pat. No. 6,193,710, the content of which is incorporated herein by reference for all purposes.

### BACKGROUND OF THE INVENTION

In general, embodiments of the present invention relate the field of vision treatment. Exemplary embodiments relate to systems and methods for providing annular and other ablation profiles for refractive surgery.

Many current laser correction techniques use small spot scanning systems or broad beam lasers for treating a wide variety of vision conditions, such as myopia and hyperopia. Although these and other proposed treatment devices and methods may provide real benefits to patients in need thereof, still further advances would be desirable. For example, there continues to be a need for improved treatment systems and methods that provide enhanced efficiency. Embodiments of the present invention provide solutions that address certain inefficiencies or shortcomings which may be associated with known techniques, and hence provide answers to at least some of these outstanding needs.

### BRIEF SUMMARY OF THE INVENTION

It has been discovered that use of a general basis data framework that allows the implementation of both circular and annular ablation profiles can increase ablation efficiency when treating certain vision conditions. Embodiments of the present invention provide techniques for using annular and other ablation profiles during refractive surgery treatment procedures. These techniques can be implemented in a variety of laser devices, including without limitation the VISX WaveScan WaveFront® System and VISX STAR S4® Excimer Laser System, the Wavelight® Alegretto laser, the Alcon Ladarvision® lasers, the Bausch and Lomb Zyoptix® laser, the Zeiss® laser, and the like.

With some current vision treatment systems, the time involved for carrying out particular procedures can vary according to the vision condition addressed. As an example, for some laser systems it takes longer to perform a hyperopic treatment than it does to perform a myopic treatment. In instances where the duration of treatment time is excessively lengthy, clinical results may be less than optimal, in part because the eye tissue may undergo substantial dehydration during the course of treatment.

The use of annular ablation profiles can effectively remove tissue during an ablation procedure which delivers a hyperopic treatment shape to the patient. When compared with some existing or known techniques, the use of annular ablation profiles can increase the ablation efficiency for hyperopic ablations by a factor of 2 to 10, for example, depending on the actual refraction. The use of symmetric and asymmetric ablations shapes such as double spots, triple spots, quadruple spots, multiple spots, arc shapes, elliptical shapes, and the like, can be extended to cover a wide spectrum of shapes.

Embodiments further encompass techniques for switching between circular and annular spot shapes during administration of a treatment. Relatedly, a mechanical block approach may involve pre-sorting the pulses in such a way that most or all of the annular pulses with the same block size are adjacent to reduce the switching. A DOE approach may involve instantaneous switching.

In one aspect, embodiments of the present invention encompass systems for ablating optical tissue in an eye of a patient. Exemplary systems include a laser mechanism that generates multiple laser beam pulses, such that each laser beam pulse has an original geometry, and a mechanical block mechanism that transforms the laser beam pulses, such that each transformed laser beam pulse has an annular geometry. The mechanical block mechanism may include an adjustable iris mechanism and a central block mechanism including one or more obscuration elements that can be positioned along a laser beam path and aligned with the adjustable iris mechanism to define an annular shaped passage. Systems may also include a delivery mechanism that directs the transformed laser beam pulses toward the eye of the patient so as to ablate the optical tissue. An obscuration element can be positionable to block an inner portion of the original geometry laser beam pulse, and an outer portion of the original geometry laser beam pulse transmitted through the annular shaped passage can provide the transformed laser beam pulse having the annular geometry.

In some cases, the central block mechanism is configured to rotate about an axis, such that the one or more obscuration elements are independently positionable relative to the adjustable iris mechanism to define the annular shaped passage. In some cases, the central block mechanism defines an obscuration blank positionable relative to the adjustable iris mechanism to define a circular shaped passage therethrough. Relatedly, the central block mechanism can include an obscuration blank positionable relative to the adjustable iris mechanism, such that the adjustable iris mechanism in combination with the obscuration blank provides a circular shaped passage for transmission of the laser beam pulses. Optionally, the adjustable iris mechanism can be adjustable to an outer diameter that is 1.0 mm, 1.5 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, or 10.0 mm. In some instances, the adjustable iris mechanism is adjustable to an outer diameter within a range from about 1.0 mm to about 10.0 mm. The one or more obscuration elements can present a mechanical block inner diameter within a range from about 2.625 mm to about 7.5 mm. In some instances, the adjustable iris mechanism defines a mechanical block outer diameter and the one or more obscuration elements define a mechanical block inner diameter, such that an obscuration ratio calculated by dividing the inner diameter by the outer diameter is about 0.75.

In another aspect, embodiments of the present invention provide methods for ablating optical tissue in an eye of a patient. Exemplary methods can include generating multiple laser beam pulses with a laser, such that each laser beam pulse has an original geometry, and transforming the laser beam pulses with a mechanical block mechanism, such that each transformed laser beam pulse having an annular geometry. Methods may also include directing the transformed laser beam pulses toward the eye of the patient so as to ablate the optical tissue. In some cases, the directing step includes scanning the transformed laser beam pulses toward the eye in a nondeterministic pattern. In some cases, the annular geometry is determined based on an optimal obscuration ratio. Optionally, the transforming step can be performed based on an outer dimension of the laser beam pulse. In some cases, the annular geometry includes an outer dimension that is determined based on an optical zone of the patient.

In still another aspect, embodiments of the present invention include system for ablating optical tissue in an eye of a patient. Systems may include a laser mechanism that generates multiple laser beam pulses, such that each laser beam pulse has an original geometry, and a mechanical block mechanism that transforms the laser beam pulses, such that each transformed laser beam pulse having an annular geometry. Systems may also include a delivery mechanism that directs the transformed laser beam pulses toward the eye of the patient so as to ablate the optical tissue. In some instances, the delivery mechanism is configured to scan the transformed laser beam pulses toward the eye in a nondeterministic pattern. In some instances, the mechanical block mechanism is configured to transform the laser beam pulses to the annular geometry based on an optimal obscuration ratio. According to some embodiments, the mechanical block mechanism can be configured to transform the laser beam pulses to the annular geometry based on an outer dimension of the laser beam pulse. Optionally, the mechanical block mechanism can be configured to transform the laser beam pulses to the annular geometry having an outer dimension that is determined based on an optical zone of the patient. In some cases, the delivery mechanism can be configured to direct the transformed laser beam pulses toward the eye of the patient in a nonconcentric pattern. An exemplary mechanical block mechanism can include an adjustable iris mechanism and a central block mechanism. The central block mechanism may include one or more obscuration elements that can be positioned along a laser beam path and aligned with the adjustable iris mechanism to define an annular shaped passage. In some instances, the one or more obscuration elements are independently positionable relative to the adjustable iris mechanism to block an inner portion of the original geometry laser beam pulse, and an outer portion of the original geometry laser beam pulse transmitted through the annular shaped passage provides the transformed laser beam pulse having the annular geometry.

In yet another aspect, embodiments of the present invention encompass methods for ablating optical tissue in an eye of a patient, which include for example generating multiple laser beam pulses with a laser, such that each laser beam pulse has an original geometry, and transforming the laser beam pulses with a transformation mechanism, such that each transformed laser beam pulse having an annular geometry. Methods may also include scanning the transformed laser beam pulses toward the eye in a nondeterministic pattern so as to ablate the optical tissue. Exemplary methods for ablating optical tissue in an eye of a patient may include generating multiple laser beam pulses with a laser such that each laser beam pulse has an original geometry, transforming the laser beam pulses with a transformation mechanism such that each transformed laser beam pulse having an annular geometry, and scanning the transformed laser beam pulses toward the eye such that a first transformed annular laser beam pulse and a second transformed annular laser beam pulse are directed toward the eye in a nonconcentric pattern. Some exemplary methods for ablating optical tissue in an eye of a patient may include generating multiple laser beam pulses with a laser such that each laser beam pulse has an original geometry, transforming the laser beam pulses with a transformation mechanism such that each transformed laser beam pulse has an annular geometry that is based on an optimal obscuration ratio, and directing the transformed laser beam pulses toward the eye of the patient so as to ablate the optical tissue. In some embodiments, the optimal obscuration ratio is based on an inner obscuration diameter within a range from about 1 mm to about 4 mm. In some embodiments, the optimal obscuration ratio is based on a variable inner radius and a variable outer radius. In some instances, the optimal obscuration ratio is within a range from about 0.68 to about 0.80. In some instances, the optimal obscuration ratio is about 0.75.

In another aspect, embodiments of the present invention encompass methods for ablating optical tissue in an eye of a patient that include receiving an ablation pulse outer dimension parameter, generating multiple laser beam pulses with a laser such that each laser beam pulse having an original geometry, and transforming one or more laser beam pulse of the multiple laser beam pulses with a transformation mechanism if the outer dimension parameter meets or exceeds a transformation threshold, such that the transformed first laser beam pulse has an annular geometry. Methods may also include directing any original geometry laser beam pulses and any transformed annular laser beam pulses toward the eye of the patient so as to ablate the optical tissue. In some instances, the transformation threshold can be within a range from about 2.5 mm to about 3 mm. In some instances, the transformation threshold can be determined based on a refraction prescription for the patient. Optionally, the transformation threshold can be determined based on an optical zone of the patient.

In some aspects, embodiments of the present invention encompass methods for ablating optical tissue in an eye of a patient that include receiving an optical zone parameter of a patient, generating multiple laser beam pulses with a laser such that each laser beam pulse has an original geometry, transforming the laser beam pulses with a transformation mechanism such that each transformed laser beam pulse has an annular geometry defined by an outer dimension parameter that is based on the optical zone parameter of the patient, and directing the transformed laser beam pulses toward the eye of the patient so as to ablate the optical tissue.

In another aspect, embodiments of the present invention include optical tissue ablation systems that include a laser mechanism that generates multiple laser beam pulses such that each laser beam pulse has an original geometry, a transformation mechanism that transforms the laser beam pulses such that each transformed laser beam pulse has an annular geometry, and a delivery mechanism that scans the transformed laser beam pulses toward the eye of the patient in a nondeterministic pattern so as to ablate the optical tissue.

In another aspect, embodiments of the present invention include optical tissue ablation systems that include a laser mechanism that generates multiple laser beam pulses such that each laser beam pulse has an original geometry, a transformation mechanism that transforms the laser beam pulses such that each transformed laser beam pulse has an annular geometry, and a scanning mechanism that scans the transformed laser beam pulses toward the eye of the patient such that a first transformed annular laser beam pulse and a second transformed annular laser beam pulse are directed toward the eye in a nonconcentric pattern.

In another aspect, embodiments of the present invention include optical tissue ablation systems that include a laser mechanism that generates multiple laser beam pulses such that each laser beam pulse has an original geometry, a transformation mechanism that transforms the laser beam pulses such that each transformed laser beam pulse has an annular geometry that is based on an optimal obscuration ratio, and a delivery mechanism that directs the transformed laser beam pulses toward the eye of the patient so as to ablate the optical tissue.

In still another aspect, embodiments of the present invention include optical tissue ablation systems that include an input module having a tangible medium embodying machine-readable code that accepts an ablation pulse outer dimension parameter, a laser mechanism that generates multiple laser beam pulses such that each laser beam pulse has an original geometry, a transformation mechanism that transforms the laser beam pulses if the outer dimension parameter meets or exceeds a transformation threshold such that each transformed laser beam pulse has an annular geometry, and a delivery mechanism that directs the original geometry laser beam pulses and the transformed annular laser beam pulses toward the eye of the patient so as to ablate the optical tissue.

In another aspect, embodiments of the present invention include optical tissue ablation systems that include an input module having a tangible medium embodying machine-readable code that receives an optical zone parameter of a patient, a laser mechanism that generates multiple laser beam pulses such that each laser beam pulse has an original geometry, a transformation mechanism that transforms the laser beam pulses such that each transformed laser beam pulse has an annular geometry that defines an outer dimension parameter based on the optical zone parameter of the patient, and a delivery mechanism that directs the transformed laser beam pulses toward the eye of the patient so as to ablate the optical tissue.

In another aspect, embodiments encompass a computer program product for ablating optical tissue in an eye of a patient that includes code for generating multiple laser beam pulses with a laser such that each laser beam pulse has an original geometry, code for transforming the laser beam pulses with a mechanical block mechanism such that each transformed laser beam pulse has an annular geometry, and code for directing the transformed laser beam pulses toward the eye of the patient so as to ablate the optical tissue.

In still another aspect, embodiments encompass a computer program product for ablating optical tissue in an eye of a patient that includes code for generating multiple laser beam pulses with a laser such that each laser beam pulse having an original geometry, code for transforming the laser beam pulses with a transformation mechanism such that each transformed laser beam pulse having an annular geometry, and code for scanning the transformed laser beam pulses toward the eye in a nondeterministic pattern so as to ablate the optical tissue.

In yet another aspect, embodiments encompass a computer program product for ablating optical tissue in an eye of a patient that includes code for generating multiple laser beam pulses with a laser such that each laser beam pulse has an original geometry, code for transforming the laser beam pulses with a transformation mechanism such that each transformed laser beam pulse has an annular geometry, and code for scanning the transformed laser beam pulses toward the eye such that a first transformed annular laser beam pulse and a second transformed annular laser beam pulse are directed toward the eye in a nonconcentric pattern.

In still yet another aspect, embodiments encompass a computer program product for ablating optical tissue in an eye of a patient that includes code for generating multiple laser beam pulses with a laser such that each laser beam pulse has an original geometry, code for transforming the laser beam pulses with a transformation mechanism such that each transformed laser beam pulse has an annular geometry that is based on an optimal obscuration ratio, and code for directing the transformed laser beam pulses toward the eye of the patient so as to ablate the optical tissue.

In yet a further aspect, embodiments encompass a computer program product for ablating optical tissue in an eye of a patient that includes code for receiving an ablation pulse outer dimension parameter, code for generating multiple laser beam pulses with a laser such that each laser beam pulse having an original geometry, code for transforming one or more laser beam pulse of the multiple laser beam pulses with a transformation mechanism such that the transformed first laser beam pulse have an annular geometry if the outer dimension parameter meets or exceeds a transformation threshold, and code for directing any original geometry laser beam pulses and any transformed annular laser beam pulses toward the eye of the patient so as to ablate the optical tissue.

In still yet a further aspect, embodiments encompass a computer program product for ablating optical tissue in an eye of a patient that includes code for receiving an optical zone parameter of a patient, code for generating multiple laser beam pulses with a laser such that each laser beam pulse having an original geometry, code for transforming the laser beam pulses with a transformation mechanism such that each transformed laser beam pulse has an annular geometry, the annular geometry having an outer dimension parameter that is based on the optical zone parameter of the patient, and code for directing the transformed laser beam pulses toward the eye of the patient so as to ablate the optical tissue.

For a fuller understanding of the nature and advantages of the present invention, reference should be had to the ensuing detailed description taken in conjunction with the accompanying drawings.

### BRIEF DESCRIPTION OF THE DRAWINGS

FIG. 1 illustrates a laser ablation system according to an embodiment of the present invention.

FIG. 2 illustrates a simplified computer system according to an embodiment of the present invention.

FIG. 3 illustrates a wavefront measurement system according to an embodiment of the present invention.

FIG. 3A illustrates another wavefront measurement system according to an embodiment of the present invention.

FIG. 4A illustrates “Before” and “After” aspects of a hyperopia treatment according to embodiments of the present invention.

FIG. 4B shows a profile of an exemplary hyperopic treatment according to embodiments of the present invention.

FIG. 4C depicts top views of a circular ablation shape (left side) and an annular ablation shape (right side) according to embodiments of the present invention.

FIG. 4D depicts side views of a circular ablation shape (left side) and an annular ablation shape (right side) according to embodiments of the present invention.

FIG. 5 provides an illustration of various shapes which can be used in a variable ring scanning (VRS) technique, according to embodiments of the present invention.

FIG. 6 shows tissue basis data according to embodiments of the present invention.

FIG. 7A shows a schematic for the buildup of annular pulses (left panel) compared to the administration of circular pulses (right panel) for a hyperopia treatment, according to embodiments of the present invention.

FIG. 7B shows a schematic for the buildup of circular pulses for a myopia treatment, according to embodiments of the present invention.

FIG. 8 shows a comparison of the number of pulses (left panel) and treatment time (right panel) between the theoretical model and the actual numbers for myopia, according to embodiments of the present invention.

FIG. 9 depicts a comparison of the number of pulses (left panel) and treatment time (right panel) between two techniques (layer by layer; optical generation) using annular shapes and the actual numbers using circular spots for hyperopia, according to embodiments of the present invention.

FIG. 10 shows a comparison of the number of pulses (left panel) and treatment time (right panel) for various maximum spot sizes of 5 mm, 4 mm, and 3 mm for myopia, according to embodiments of the present invention.

FIG. 11 illustrates a comparison of the number of pulses (left panel) and treatment time (right panel) for various maximum spot sizes of 5 mm, 4 mm, and 3 mm for hyperopia, according to embodiments of the present invention.

FIG. 12 shows a relationship between maximum iris size and maximum OZ according to embodiments of the present invention.

FIG. 13 shows a relationship between obscuration ratio and maximum Rx (D), for 7 mm (optical zone) ×9 mm (ablation zone) and 6 mm (optical zone) ×8 mm (ablation zone), according to embodiments of the present invention.

FIG. 14 provides a graphic illustration of the relationship between ablation time and myopia, mixed, and hyperopia treatments, for 20 Hz, 35 Hz, and 50 Hz repetition rates, for example as evaluated through a Monte Carlo simulation, according to embodiments of the present invention.

FIG. 15 shows a relationship between the ablation depth and distance from the iris center, according to embodiments of the present invention.

FIG. 16 shows a relationship between ablation depth and distance from iris center, according to embodiments of the present invention.

FIG. 17 shows a relationship between ablation depth and distance from iris center, according to embodiments of the present invention.

FIG. 18 shows aspects of a mechanical block assembly according to embodiments of the present invention.

FIG. 19 shows an optical set up, according to embodiments of the present invention.

FIG. 20 shows a comparison of ablation times for 6 mm (optical zone) ×9 mm (ablation zone) circular and 6 mm (optical zone) ×8 mm (ablation zone) annular embodiments, according to embodiments of the present invention.

FIG. 21 shows a comparison of ablation times for 7 mm (optical zone) ×9 mm (ablation zone) embodiments, according to embodiments of the present invention.

FIG. 22 depicts a relationship between ablation time and pre operative refraction, for myopia and hyperopia, according to embodiments of the present invention.

FIG. 23 shows ablation times for a +3 D hyperopia treatment according to embodiments of the present invention.

FIG. 24 illustrates a decision tree or method associated with an intended approval range for refractive treatments, or with approaches for building a treatment system, according to embodiments of the present invention.

### DETAILED DESCRIPTION OF THE INVENTION

The broad beam top hat laser profile of ablation systems such as Visx STAR systems are highly effective in ablating myopic shapes, due to the high efficiency of material removal in unit time. It has been discovered that similar efficiencies can be achieved for the ablation of hyperopic shapes. For example reducing the maximum spot size from 6.5 mm to about 4 mm, can effectively reducing the maximum efficiency to 4²/6.5²=38%. Furthermore, the solution accuracy tolerance, which may be defined as the root mean squares (RMS) error between a target shape and an ablated shape, can involve the use of more small pulses, bringing such an efficiency reduction in practice to the level of nearly 15% for hyperopia. For example, a typical −4 D treatment may involve an ablation of 20 seconds, and a typical +4 treatment may involve an ablation of 120 seconds to ablation, with a 20 Hz laser. The use of annular ablation shapes optionally combined with circular ablation shapes can improve the treatment time for hyperopia.

Embodiments of the present invention can be readily adapted for use with existing laser systems and other optical treatment devices. Although system, software, and method embodiments of the present invention are described primarily in the context of a laser eye surgery system, it should be understood that embodiments of the present invention may be adapted for use in alternative eye treatment procedures, systems, or modalities, such as spectacle lenses, intraocular lenses, accommodating IOLs, contact lenses, corneal ring implants, collagenous corneal tissue thermal remodeling, corneal inlays, corneal onlays, other corneal implants or grafts, and the like. Relatedly, systems, software, and methods according to embodiments of the present invention are well suited for customizing any of these treatment modalities to a specific patient. Thus, for example, embodiments encompass custom intraocular lenses, custom contact lenses, custom corneal implants, and the like, which can be configured to treat or ameliorate any of a variety of vision conditions in a particular patient based on their unique ocular characteristics or anatomy.

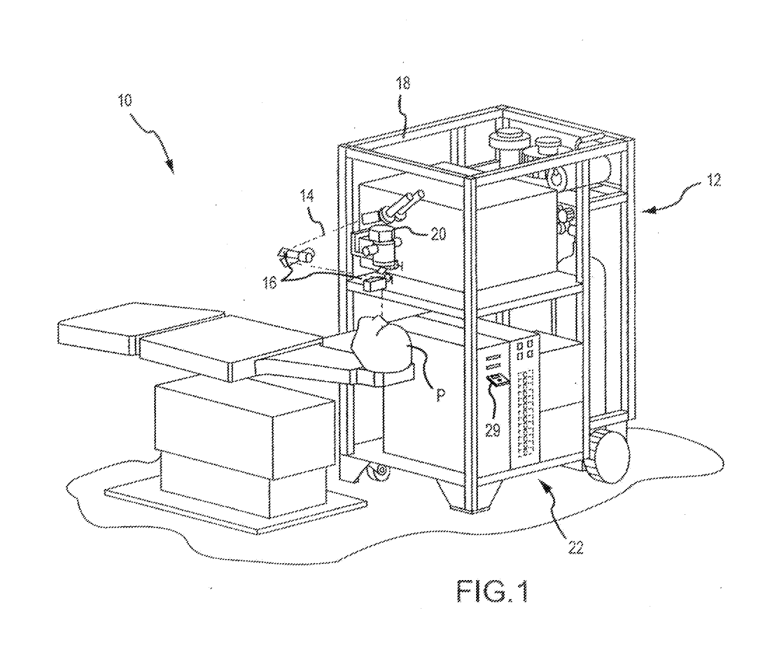

Turning now to the drawings, FIG. 1 illustrates a laser eye surgery system 10 of the present invention, including a laser 12 that produces a laser beam 14. Laser 12 is optically coupled to laser delivery optics 16, which directs laser beam 14 to an eye E of patient P. A delivery optics support structure (not shown here for clarity) extends from a frame 18 supporting laser 12. A microscope 20 is mounted on the delivery optics support structure, the microscope often being used to image a cornea of eye E.

Laser 12 generally comprises an excimer laser, ideally comprising an argon-fluorine laser producing pulses of laser light having a wavelength of approximately 193 nm. Laser 12 will preferably be designed to provide a feedback stabilized fluence at the patient's eye, delivered via delivery optics 16. The present invention may also be useful with alternative sources of ultraviolet or infrared radiation, particularly those adapted to controllably ablate the corneal tissue without causing significant damage to adjacent and/or underlying tissues of the eye. Such sources include, but are not limited to, solid state lasers and other devices which can generate energy in the ultraviolet wavelength between about 185 and 205 nm and/or those which utilize frequency-multiplying techniques. Hence, although an excimer laser is the illustrative source of an ablating beam, other lasers may be used in the present invention.

Laser system 10 will generally include a computer or programmable processor 22. Processor 22 may comprise (or interface with) a conventional PC system including the standard user interface devices such as a keyboard, a display monitor, and the like. Processor 22 will typically include an input device such as a magnetic or optical disk drive, an internet connection, or the like. Such input devices will often be used to download a computer executable code from a tangible storage media 29 embodying any of the methods of the present invention. Tangible storage media 29 may take the form of a floppy disk, an optical disk, a data tape, a volatile or non-volatile memory, RAM, or the like, and the processor 22 will include the memory boards and other standard components of modern computer systems for storing and executing this code. Tangible storage media 29 may optionally embody wavefront sensor data, wavefront gradients, a wavefront elevation map, a treatment map, a corneal elevation map, and/or an ablation table. While tangible storage media 29 will often be used directly in cooperation with a input device of processor 22, the storage media may also be remotely operatively coupled with processor by means of network connections such as the internet, and by wireless methods such as infrared, Bluetooth, or the like.

Laser 12 and delivery optics 16 will generally direct laser beam 14 to the eye of patient P under the direction of a computer 22. Computer 22 will often selectively adjust laser beam 14 to expose portions of the cornea to the pulses of laser energy so as to effect a predetermined sculpting of the cornea and alter the refractive characteristics of the eye. In many embodiments, both laser beam 14 and the laser delivery optical system 16 will be under computer control of processor 22 to effect the desired laser sculpting process, with the processor effecting (and optionally modifying) the pattern of laser pulses. The pattern of pulses may by summarized in machine readable data of tangible storage media 29 in the form of a treatment table, and the treatment table may be adjusted according to feedback input into processor 22 from an automated image analysis system in response to feedback data provided from an ablation monitoring system feedback system. Optionally, the feedback may be manually entered into the processor by a system operator. Such feedback might be provided by integrating the wavefront measurement system described below with the laser treatment system 10, and processor 22 may continue and/or terminate a sculpting treatment in response to the feedback, and may optionally also modify the planned sculpting based at least in part on the feedback. Measurement systems are further described in U.S. Pat. No. 6,315,413, the full disclosure of which is incorporated herein by reference.

Laser beam 14 may be adjusted to produce the desired sculpting using a variety of alternative mechanisms. The laser beam 14 may be selectively limited using one or more variable apertures. An exemplary variable aperture system having a variable iris and a variable width slit is described in U.S. Pat. No. 5,713,892, the full disclosure of which is incorporated herein by reference. The laser beam may also be tailored by varying the size and offset of the laser spot from an axis of the eye, as described in U.S. Pat. Nos. 5,683,379, 6,203,539, and 6,331,177, the full disclosures of which are incorporated herein by reference.

Still further alternatives are possible, including scanning of the laser beam over the surface of the eye and controlling the number of pulses and/or dwell time at each location, as described, for example, by U.S. Pat. No. 4,665,913, the full disclosure of which is incorporated herein by reference; using masks in the optical path of laser beam 14 which ablate to vary the profile of the beam incident on the cornea, as described in U.S. Pat. No. 5,807,379, the full disclosure of which is incorporated herein by reference; hybrid profile-scanning systems in which a variable size beam (typically controlled by a variable width slit and/or variable diameter iris diaphragm) is scanned across the cornea; or the like. The computer programs and control methodology for these laser pattern tailoring techniques are well described in the patent literature.

Additional components and subsystems may be included with laser system 10, as should be understood by those of skill in the art. For example, spatial and/or temporal integrators may be included to control the distribution of energy within the laser beam, as described in U.S. Pat. No. 5,646,791, the full disclosure of which is incorporated herein by reference. Ablation effluent evacuators/filters, aspirators, and other ancillary components of the laser surgery system are known in the art. Further details of suitable systems for performing a laser ablation procedure can be found in commonly assigned U.S. Pat. Nos. 4,665,913, 4,669,466, 4,732,148, 4,770,172, 4,773,414, 5,207,668, 5,108,388, 5,219,343, 5,646,791 and 5,163,934, the complete disclosures of which are incorporated herein by reference. Suitable systems also include commercially available refractive laser systems such as those manufactured and/or sold by Alcon, Bausch & Lomb, Nidek, WaveLight, LaserSight, Schwind, Zeiss-Meditec, and the like. Basis data can be further characterized for particular lasers or operating conditions, by taking into account localized environmental variables such as temperature, humidity, airflow, and aspiration.

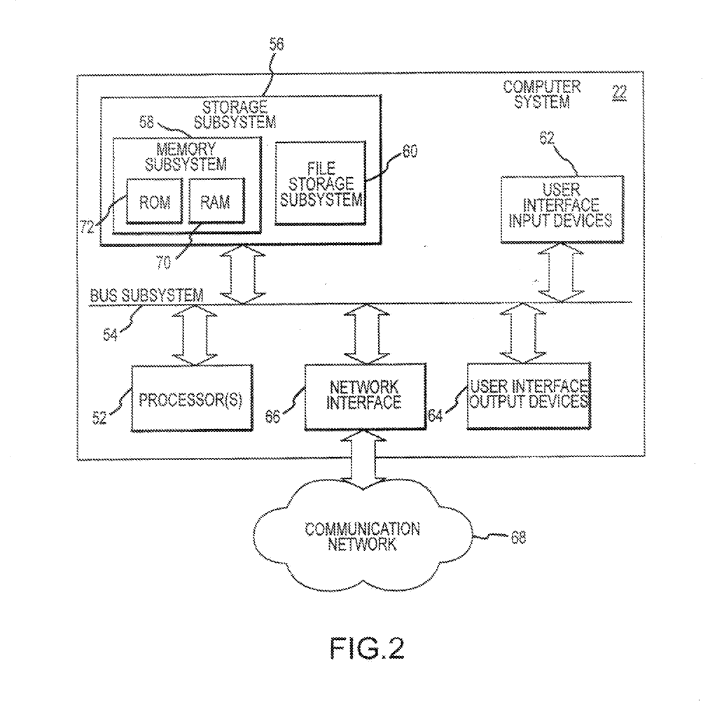

FIG. 2 is a simplified block diagram of an exemplary computer system 22 that may be used by the laser surgical system 10 of the present invention. Computer system 22 typically includes at least one processor 52 which may communicate with a number of peripheral devices via a bus subsystem 54. These peripheral devices may include a storage subsystem 56, comprising a memory subsystem 58 and a file storage subsystem 60, user interface input devices 62, user interface output devices 64, and a network interface subsystem 66. Network interface subsystem 66 provides an interface to outside networks 68 and/or other devices, such as the wavefront measurement system 30.

User interface input devices 62 may include a keyboard, pointing devices such as a mouse, trackball, touch pad, or graphics tablet, a scanner, foot pedals, a joystick, a touchscreen incorporated into the display, audio input devices such as voice recognition systems, microphones, and other types of input devices. User input devices 62 will often be used to download a computer executable code from a tangible storage media 29 embodying any of the methods of the present invention. In general, use of the term “input device” is intended to include a variety of conventional and proprietary devices and ways to input information into computer system 22.

User interface output devices 64 may include a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide a non-visual display such as via audio output devices. In general, use of the term “output device” is intended to include a variety of conventional and proprietary devices and ways to output information from computer system 22 to a user.

Storage subsystem 56 can store the basic programming and data constructs that provide the functionality of the various embodiments of the present invention. For example, a database and modules implementing the functionality of the methods of the present invention, as described herein, may be stored in storage subsystem 56. These software modules are generally executed by processor 52. In a distributed environment, the software modules may be stored on a plurality of computer systems and executed by processors of the plurality of computer systems. Storage subsystem 56 typically comprises memory subsystem 58 and file storage subsystem 60.

Memory subsystem 58 typically includes a number of memories including a main random access memory (RAM) 70 for storage of instructions and data during program execution and a read only memory (ROM) 72 in which fixed instructions are stored. File storage subsystem 60 provides persistent (non-volatile) storage for program and data files, and may include tangible storage media 29 (FIG. 1) which may optionally embody wavefront sensor data, wavefront gradients, a wavefront elevation map, a treatment map, and/or an ablation table. File storage subsystem 60 may include a hard disk drive, a floppy disk drive along with associated removable media, a Compact Digital Read Only Memory (CD-ROM) drive, an optical drive, DVD, CD-R, CD-RW, solid-state removable memory, and/or other removable media cartridges or disks. One or more of the drives may be located at remote locations on other connected computers at other sites coupled to computer system 22. The modules implementing the functionality of the present invention may be stored by file storage subsystem 60.

Bus subsystem 54 provides a mechanism for letting the various components and subsystems of computer system 22 communicate with each other as intended. The various subsystems and components of computer system 22 need not be at the same physical location but may be distributed at various locations within a distributed network. Although bus subsystem 54 is shown schematically as a single bus, alternate embodiments of the bus subsystem may utilize multiple busses.

Computer system 22 itself can be of varying types including a personal computer, a portable computer, a workstation, a computer terminal, a network computer, a control system in a wavefront measurement system or laser surgical system, a mainframe, or any other data processing system. Due to the ever-changing nature of computers and networks, the description of computer system 22 depicted in FIG. 2 is intended only as a specific example for purposes of illustrating one embodiment of the present invention. Many other configurations of computer system 22 are possible having more or less components than the computer system depicted in FIG. 2.

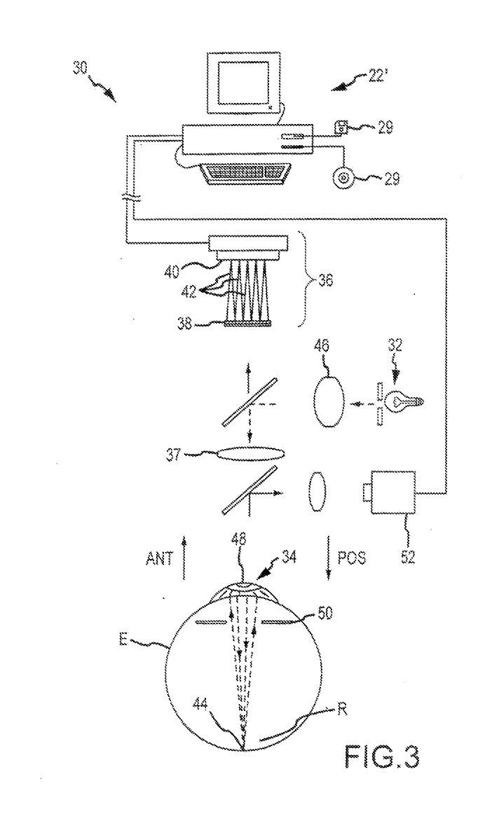

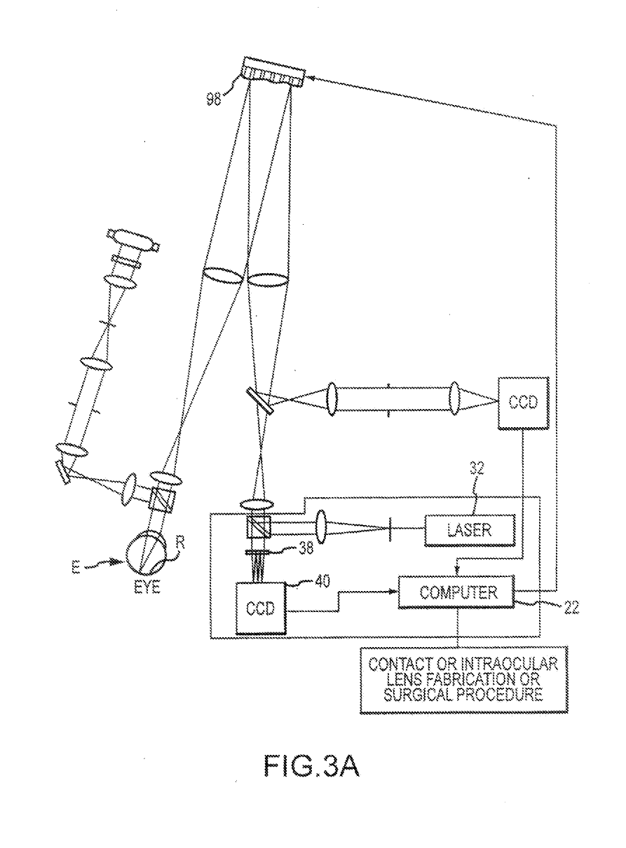

Referring now to FIG. 3, one embodiment of a wavefront measurement system 30 is schematically illustrated in simplified form. In very general terms, wavefront measurement system 30 is configured to sense local slopes of a gradient map exiting the patient's eye. Devices based on the Hartmann-Shack principle generally include a lenslet array to sample the gradient map uniformly over an aperture, which is typically the exit pupil of the eye. Thereafter, the local slopes of the gradient map are analyzed so as to reconstruct the wavefront surface or map.

More specifically, one wavefront measurement system 30 includes an image source 32, such as a laser, which projects a source image through optical tissues 34 of eye E so as to form an image 44 upon a surface of retina R. The image from retina R is transmitted by the optical system of the eye (e.g., optical tissues 34) and imaged onto a wavefront sensor 36 by system optics 37. The wavefront sensor 36 communicates signals to a computer system 22′ for measurement of the optical errors in the optical tissues 34 and/or determination of an optical tissue ablation treatment program. Computer 22′ may include the same or similar hardware as the computer system 22 illustrated in FIGS. 1 and 2. Computer system 22′ may be in communication with computer system 22 that directs the laser surgery system 10, or some or all of the components of computer system 22, 22′ of the wavefront measurement system 30 and laser surgery system 10 may be combined or separate. If desired, data from wavefront sensor 36 may be transmitted to a laser computer system 22 via tangible media 29, via an I/O port, via an networking connection 66 such as an intranet or the Internet, or the like.

Wavefront sensor 36 generally comprises a lenslet array 38 and an image sensor 40. As the image from retina R is transmitted through optical tissues 34 and imaged onto a surface of image sensor 40 and an image of the eye pupil P is similarly imaged onto a surface of lenslet array 38, the lenslet array separates the transmitted image into an array of beamlets 42, and (in combination with other optical components of the system) images the separated beamlets on the surface of sensor 40. Sensor 40 typically comprises a charged couple device or “CCD,” and senses the characteristics of these individual beamlets, which can be used to determine the characteristics of an associated region of optical tissues 34. In particular, where image 44 comprises a point or small spot of light, a location of the transmitted spot as imaged by a beamlet can directly indicate a local gradient of the associated region of optical tissue.

Eye E generally defines an anterior orientation ANT and a posterior orientation POS. Image source 32 generally projects an image in a posterior orientation through optical tissues 34 onto retina R as indicated in FIG. 3. Optical tissues 34 again transmit image 44 from the retina anteriorly toward wavefront sensor 36. Image 44 actually formed on retina R may be distorted by any imperfections in the eye's optical system when the image source is originally transmitted by optical tissues 34. Optionally, image source projection optics 46 may be configured or adapted to decrease any distortion of image 44.

In some embodiments, image source optics 46 may decrease lower order optical errors by compensating for spherical and/or cylindrical errors of optical tissues 34. Higher order optical errors of the optical tissues may also be compensated through the use of an adaptive optic element, such as a deformable mirror (described below). Use of an image source 32 selected to define a point or small spot at image 44 upon retina R may facilitate the analysis of the data provided by wavefront sensor 36. Distortion of image 44 may be limited by transmitting a source image through a central region 48 of optical tissues 34 which is smaller than a pupil 50, as the central portion of the pupil may be less prone to optical errors than the peripheral portion. Regardless of the particular image source structure, it will be generally be beneficial to have a well-defined and accurately formed image 44 on retina R.

In one embodiment, the wavefront data may be stored in a computer readable medium 29 or a memory of the wavefront sensor system 30 in two separate arrays containing the x and y wavefront gradient values obtained from image spot analysis of the Hartmann-Shack sensor images, plus the x and y pupil center offsets from the nominal center of the Hartmann-Shack lenslet array, as measured by the pupil camera 51 (FIG. 3) image. Such information contains all the available information on the wavefront error of the eye and is sufficient to reconstruct the wavefront or any portion of it. In such embodiments, there is no need to reprocess the Hartmann-Shack image more than once, and the data space required to store the gradient array is not large. For example, to accommodate an image of a pupil with an 8 mm diameter, an array of a 20×20 size (i.e., 400 elements) is often sufficient. As can be appreciated, in other embodiments, the wavefront data may be stored in a memory of the wavefront sensor system in a single array or multiple arrays.

While the methods of the present invention will generally be described with reference to sensing of an image 44, a series of wavefront sensor data readings may be taken. For example, a time series of wavefront data readings may help to provide a more accurate overall determination of the ocular tissue aberrations. As the ocular tissues can vary in shape over a brief period of time, a plurality of temporally separated wavefront sensor measurements can avoid relying on a single snapshot of the optical characteristics as the basis for a refractive correcting procedure. Still further alternatives are also available, including taking wavefront sensor data of the eye with the eye in differing configurations, positions, and/or orientations. For example, a patient will often help maintain alignment of the eye with wavefront measurement system 30 by focusing on a fixation target, as described in U.S. Pat. No. 6,004,313, the full disclosure of which is incorporated herein by reference. By varying a position of the fixation target as described in that reference, optical characteristics of the eye may be determined while the eye accommodates or adapts to image a field of view at a varying distance and/or angles.

The location of the optical axis of the eye may be verified by reference to the data provided from a pupil camera 52. In the exemplary embodiment, a pupil camera 52 images pupil 50 so as to determine a position of the pupil for registration of the wavefront sensor data relative to the optical tissues.

An alternative embodiment of a wavefront measurement system is illustrated in FIG. 3A. The major components of the system of FIG. 3A are similar to those of FIG. 3. Additionally, FIG. 3A includes an adaptive optical element 53 in the form of a deformable mirror. The source image is reflected from deformable mirror 98 during transmission to retina R, and the deformable mirror is also along the optical path used to form the transmitted image between retina R and imaging sensor 40. Deformable mirror 98 can be controllably deformed by computer system 22 to limit distortion of the image formed on the retina or of subsequent images formed of the images formed on the retina, and may enhance the accuracy of the resultant wavefront data. The structure and use of the system of FIG. 3A are more fully described in U.S. Pat. No. 6,095,651, the full disclosure of which is incorporated herein by reference.

The components of an embodiment of a wavefront measurement system for measuring the eye and ablations may comprise elements of a WaveScan® system, available from VISX, INCORPORATED of Santa Clara, Calif. One embodiment includes a WaveScan system with a deformable mirror as described above. An alternate embodiment of a wavefront measuring system is described in U.S. Pat. No. 6,271,915, the full disclosure of which is incorporated herein by reference. It is appreciated that any wavefront aberrometer could be employed for use with the present invention. Relatedly, embodiments of the present invention encompass the implementation of any of a variety of optical instruments provided by WaveFront Sciences, Inc., including the COAS wavefront aberrometer, the ClearWave contact lens aberrometer, the Crystal Wave IOL aberrometer, and the like.

Annular and Other Ablation Profiles for Refractive Surgery

With some current vision treatment systems, hyperopic ablation protocols use relatively smaller laser ablation profiles during the treatment as compared with myopic ablation protocols. Hence, the time involved for providing a hyperopic treatment can be much longer than the time involved for providing a myopic treatment, and the hyperopic treatment can be lag the myopic treatment in terms of ablation efficiency. Relatedly, at least partly due to tissue dehydration which may occur during the course of treatment, clinical results achieved in hyperopic treatments may not match the clinical results achieved in myopic treatments.

For example, for current lasers such as a VISX STAR S4® Excimer Laser System operating at 20 Hz with a 6.5 mm maximum spot size, the treatment time for hyperopia is on average about six (6) times that for myopia, for the same refractive power. This is due to the fact that the hyperopic ablation shape is donut-like and the maximum spot size used for myopia is about 4 mm, compared to 6.5 mm for myopia. It has been discovered that by using annular ablation shapes, a maximum reduction factor of 3.1 can be achieved in treatment time for hyperopia. If the annular shapes are used in a way to preserve energy, the reduction factor can be as high as 3.7, making it 1.5 to 2 times the corresponding myopic ablation time.

As another example, for faster or higher repetition rate lasers, which may include broad beam lasers, operating at 50 Hz with 5 mm, 4 mm, and 3 mm maximum spot sizes, the average treatment time for myopia is 85%, 150%, and 185% of the actual time with an such lasers for a 6.5 mm maximum spot size, respectively, without the use of rigorous thermal modeling. For hyperopia, these numbers are 150%, 120%, and 110%, respectively, when the constant fluence annular basis data are used. According to some embodiments, from an ablation efficiency viewpoint, a 5 mm maximum spot size appears to be an optimal choice.

Embodiments of the present invention provide improved ablation profiles which can significantly enhance the efficiency of vision treatment systems, including refractive surgery laser systems and the like. With small spot scanning laser treatment systems, it may be possible to drive the treatment time lower by increasing the speed of the laser repetition. With broad beam laser treatment systems, however, it may be difficult to realize similar reductions in treatment time by increasing laser repetition, particularly when treating certain vision conditions, due to the stronger energy which is associated with a broad beam (e.g. as compared with the small beam geometry). For example, as noted above, treatment times required for administering broad beam hyperopic ablation treatments may be greater than treatment times required for administering broad beam myopic ablation treatments. In some instances, implementation of an annular approach for exemplary excimer laser embodiments can help to achieve treatment times for hyperopia that are as fast as those for myopia. Such outcomes can be achieved without significant increases in the laser repetition rate.

It has been discovered that use of a general basis data framework that allows the implementation of both circular and annular ablation profiles can increase ablation efficiency when treating certain vision conditions using broad beam techniques. In some embodiments, the framework can increase the ablation efficiency of a broad beam hyperopic ablation so as to approach the ablation efficiency of a broad beam myopic ablation. Use of a general basis data framework can allow the implementation of both circular and annular ablation shapes or profiles. In some instances, a circular ablation shape or profile can be considered to present a special case of an annular ablation shape or profile. Embodiments of the present invention can provide, for example, five-fold increases in speed for hyperopia treatments with the combination of an annular shape and a repetition rate increase. In some cases, over three-fold increases in speed can be achieved, even without an increase in the repetition rate.

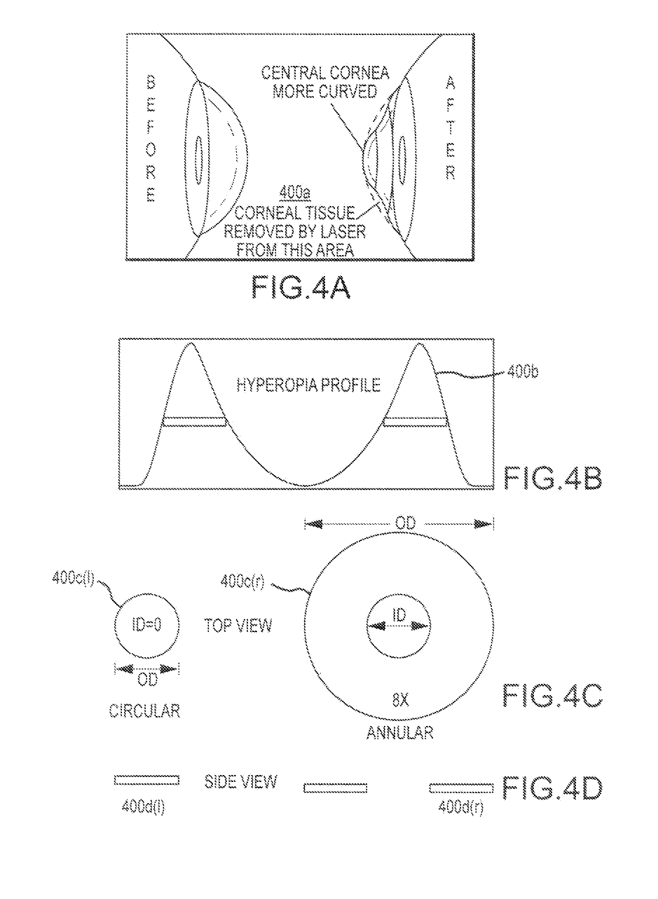

Embodiments of the present invention encompass systems and methods that implement efficient use of a broad beam (e.g. top hat) laser profile in a refractive surgery setting. In some instances, the laser profile is configured as an annular ablation profile. FIG. 4A illustrates “Before” and “After” aspects of a hyperopia treatment according to embodiments of the present invention. As shown on the left side of the figure, the shape of the hyperopic eye is slightly curved. The hyperopia treatment involves removing an annular ring of tissue 400a from the eye, which increases or steepens the curvature at the center of the cornea as shown in the treated eye on the right side of the figure.

FIG. 4B shows a profile 400b of an exemplary hyperopic treatment, which can be used when applying an annular or donut-shaped ablation to the eye. FIG. 4C depicts top views of a circular ablation shape 400c(l) (left side) and an annular ablation shape 400c(r) (right side). Relatedly, FIG. 4D depicts side views of a circular ablation shape 400d(l) (left side) and an annular ablation shape 400d(r) (right side). In some instances, a vision treatment involves the application of a number of small circular ablation spots, the cumulative effect of which is to fill a hyperopia profile such as that shown in FIG. 4B. Embodiments of the present invention also encompass systems and methods for administering vision treatments that involve the application of a number of annular ablation rings, again the cumulative effect of which is to fill a hyperopia profile such as that shown in FIG. 4B. As shown in FIG. 4C, an annular ablation shape 400c(r) can have an inner or obscuration diameter (ID) and an outer diameter (OD). The inner diameter (or radius) and the outer diameter (or radius) of the ablation pulse or profile shape can be used to determine the obscuration ratio of the shape. For example, the obscuration ratio can be calculated as the ratio of the inner or obscuration diameter (ID) to the outer diameter (OD). In instances where the inner diameter is zero, and hence the obscuration ratio is zero, the result is a circular spot or shape, as depicted in the left panel of FIG. 4C. In instances where the inner diameter is greater than zero, and hence the obscuration ratio is greater than zero, the result is an annular spot or shape, as depicted in the right panel of FIG. 4C. It has been discovered that an ablation performed with one annular pulse can cover a much larger area, as compared with an ablation performed with one circular spot pulse. For example, for the pulse sizes shown in FIGS. 4C and 4D, the area covered or the volume amount of tissue ablated with one annular pulse is eight (8) times the area covered or the volume amount of tissue ablated with one circular spot. Moreover, ablation protocols involving the application of annular pulses can result in a lower number of profile gaps as compared with ablation protocols involving the application of circular spot pulses. Hence, annular pulse ablation protocols can provide enhanced efficiency for vision treatment. In some embodiments, an obscuration area can have an elliptical shape, based on eccentricity which involves a semi-width variable and a semi-length variable.

Embodiments of the present invention encompass techniques for deciding whether to use a circular spot (i.e. obscuration diameter of zero) or an annular aperture (i.e. obscuration diameter of greater than zero) based on iris size or the nature of the treatment profile. An annular aperture may be used to produce an annular pulse shape. In some cases, the inner obscuration radius is fixed. In some cases, the inner obscuration radius is adjustable. Optionally, the inner obscuration radius may be randomly selected. Embodiments also provide optimal obscuration ratios for annular shapes. In some cases, laser beams may include harmonized beams having an outer diameter within a range from about 6.5 mm to about 8.5 mm.

According to some embodiments, annular ablation pulses or beams can be applied or directed toward the eye in an overlapping fashion. In some cases, annular ablation pulses or beams can be applied or overlapped in a non-uniform manner. Annular pulse shapes can be delivered layer by layer in a regular arrangement, such as a concentric arrangement. Optionally, annular pulse shapes can be delivered as part of a variable ring scanning (VRS) technique, as described elsewhere herein.

According to some embodiments, vision treatments encompass the application of a variable ring scanning (VRS) technique which involves the use of annular pulse ablations. Optionally, annular pulses which are administered as part of the treatment can have a variable inner diameter and a variable outer diameter. Variable ring scanning (VRS) techniques are well suited for use in administering both hyperopia and myopia treatment profiles. It has been discovered that ablation efficiencies often associated with myopia treatments using variable spot scanning (VSS) techniques, which often assume that an ablation profile can vary in size as a circular shape, can also be achieved for hyperopia treatments by using variable ring scanning (VRS) techniques.

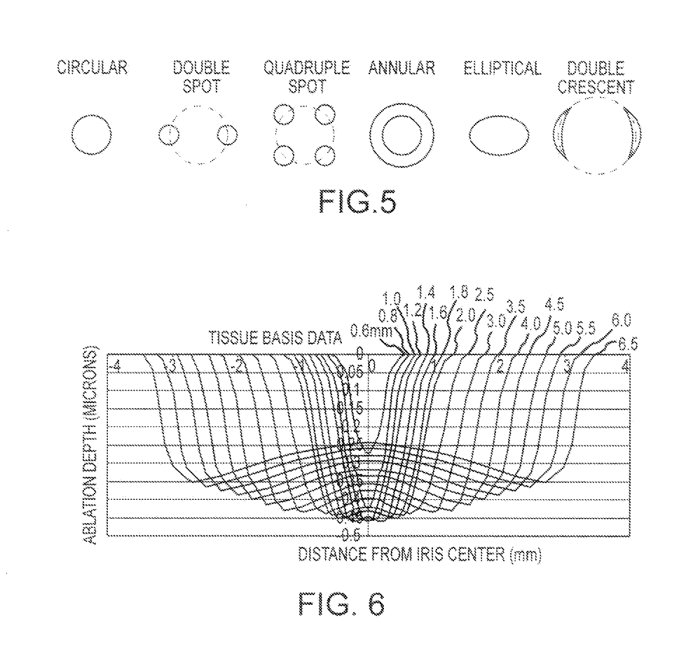

FIG. 5 provides an illustration of various shapes which can be used in a variable ring scanning (VRS) technique, including circular, double spot, quadruple spot, annular, elliptical, and double crescent ablation shapes. Embodiments of the present invention further encompass variable ring scanning (VRS) techniques which include the administration of triple spot or octal spot pulses, or various combinations of such pulse shapes, and the like.

According to some embodiments, plastic and tissue basis data can be constructed in such a way that half of the profile is provided as a pair of values (e.g. x, y) for a selected diameters such as 6.5 mm, 6 mm, and so on, down to 0.65 mm, for example in a STAR Excimer Laser System such as the VISX STAR S4® Excimer Laser System. The x variable can refer to a distance from a spot center, and the y variable can refer to the ablation depth. For a faster broad beam or higher repetition rate laser system, the maximum diameter may be about 5 mm.

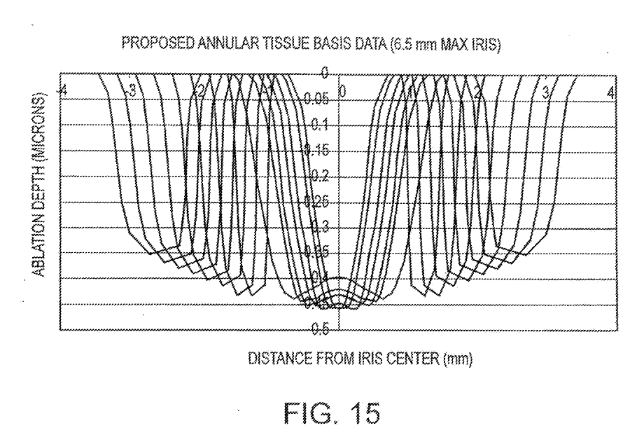

FIG. 6 shows tissue basis data according to embodiments of the present invention, illustrating an example of actual top hat basis data before any scaling, for some typical iris sizes. Often, an actual top hat is not perfectly flat. Assuming no profile change between the outer and inner diameters of a variable ring scanning pulse when a central obscuration is used, there may be no change in the basis data. In some cases, the basis data format may be redesigned to accommodate for ablation rate changes when a circular aperture changes to an annular aperture. In such instances, new architectures of three dimensional representation of the basis data may be more appropriate, because experiments have shown that for off-axis laser pulses, the ablation profile may no longer be circular. According to some embodiments, a scaling factor of 0.726 may be used for pulse depth. In some cases, it is possible to simplify the calculation or estimation, without a loss of generality, by assuming the basis data is ring type with perfectly flat bottom and square side (rectangle with side view), with both the inner and outer radius changeable with infinite resolution. FIG. 6 depicts cross-sections or side views of actual top hat laser profiles or pulses. In some cases, the terms “tissue basis” and “cross-section” may be used interchangeably. FIG. 6 pertains to circular ablation shapes or profiles, in comparison with the annular ablation shapes or profiled discussed with reference to FIG. 15.

A simulated annealing least squares algorithm (SALSA) can be used for treatment table generation. This algorithm is well suited for solving multi-dimensional inverse problems with up to millions of degree of freedom. Current implementations of the SALSA algorithm allow the availability of spots in the following ways: (1) availability of x and y scanning positions, in the resolution of 100 microns, and (2) availability of the laser spot diameter between 6.5 mm and 1 mm in the resolution of 0.25 mm. Here, the x and y variables can refer to x and y locations on a plane. In some implementations of an annular aperture technique, it is helpful to provide a range of values for the inner (obscured) diameter. For example, in certain embodiments the inner diameter can be within a range from about 1 mm to about 4 mm. It is also possible to provide a resolution or increment value for the inner diameter values. For example, in some cases the resolution or increment value can be about 1 mm, and hence the inner diameter can be adjusted to discrete settings, such as 1 mm, 2 mm, 3 mm, 4 mm, and the like.

Experiments with selected software techniques using the simulated annealing least square algorithm (SALSA) without modification, that is using a radially symmetric assumption, can cut the treatment time by half with the use of a randomly selected fixed inner obscuration radius. A modification of the algorithm, with radially symmetric pulses, to allow change of the inner radius can reduce the treatment time by more than two times. Annular as well as double spot pulses can be used, for example.

According to some embodiments, an algorithm or system can be configured to provide a decision whether to use a circular spot (i.e. obscuration diameter of zero) or an annular aperture (i.e. obscuration diameter of greater than zero), based on the nature of the treatment profile. For example, if the outer diameter or iris size is greater than about 3.0 mm, then an annular aperture can be used, and if the outer diameter or iris size is equal to or less than about 3.0 mm, then a circular aperture can be used. Other embodiments may employ different threshold or break point values. Multi-pass optimization may be implemented to further reduce the fitting error and reduce the treatment time.

In some cases, techniques can involve an extension of effective aperture and range. For example, embodiments may include a 6.5 mm maximum aperture, or larger, such as a 7 mm or 8 mm aperture. Techniques may also involve the application of a beam having uniform intensity. Optionally, hexagon optics can be used to generate an annular shape having a fixed inner:outer radius ratio, or a variable inner:outer radius ratio.

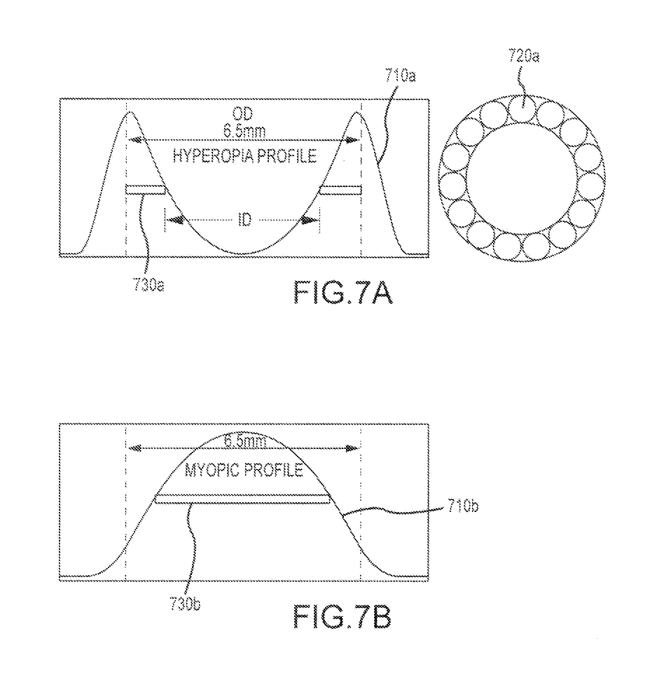

FIG. 7A shows a schematic for the buildup 710a of annular pulses (left panel) compared to the administration of circular pulses 720a (right panel) for a hyperopia treatment, to achieve similar results. With reference to the left panel, the inner diameter ID of the annular pulse can be variable, and the outer diameter OD can be set to a maximum iris diameter, such as 6.5 mm. A side view of an exemplary annular or donut shaped pulse 730a is shown. In some cases, multiple annular pulses can be administered to the eye as part of a technique which laterally shifts such pulses in an x,y scanning protocol. In this way, the cumulative buildup of annular pulses can exceed the maximum iris diameter of a single pulse. FIG. 7B shows a schematic for the buildup 710b of circular pulses for a myopia treatment. A side view of an exemplary circular shaped pulse 730b is shown.

From the real basis data, the nominal pulse depth is about 0.35 microns, corresponding to a scaled pulse depth of 0.35×0.726=0.25 microns. Calculations can be made on a layer by layer basis, with each layer corresponding to the depth of one pulse, or 0.25 microns. This can result in the bulk of the volume filled with the large pulses, either annular as shown in the left panel of FIG. 7A or circular as shown in. FIG. 7B. However, if the annular shape is generated optically, for example by a diffractive optical element mechanism, it can be designed in such a way that the energy of the original circle pulse or shape is redistributed to the subsequent annular pulse or shape, in a uniform manner without loss of energy. Due to this conservation of energy, the ablation depth per pulse will increase accordingly [check with Client: is this correct?]. Both cases, including the optical generation technique, and the layer by layer or algorithm generation technique, are considered herein.

According to some embodiments, there may be practically very little space left after the ablation of the concentric central area due to the regular shape of the cross-section of the pulse and the flexibility of inner and outer diameters of the iris. However, to fill up with the untouched space outside the maximum 6.5 mm zone, it is helpful to fill with smaller pulses. To further simplify the calculation, it is possible to assume a fill factor of 100% in the case to use small pulses to fill up rings outside the 6.5 mm zone and drop the entire gap when the spot diameter is smaller than 1 mm, the minimum spot size. To determine the ablation time, half of the maximum ablation rate can be used as the average repetition rate for myopia, and the maximum ablation rate can be used directly as the average repetition rate for hyperopia.

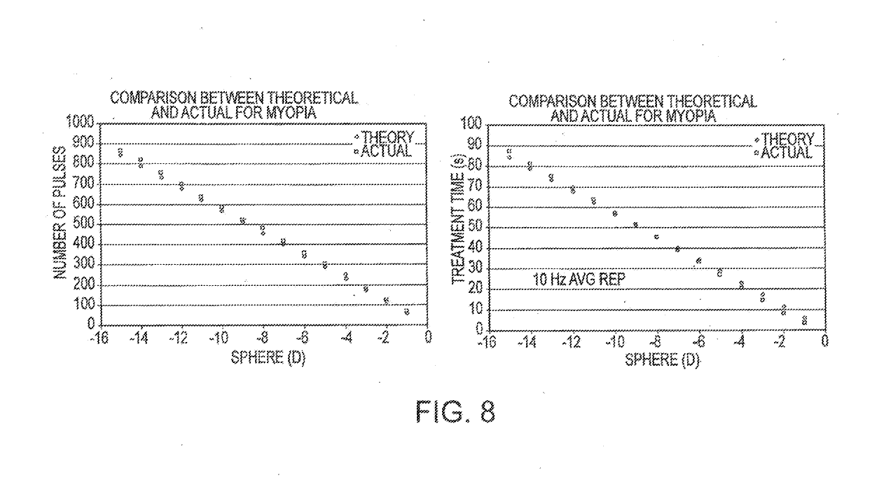

FIG. 8 provides a comparison of the number of pulses (left panel) and treatment time (right panel) between the theoretical model and the actual numbers for myopia. Specifically, FIG. 8 shows the number of pulses and treatment time for myopia between the theoretically estimated and the actual numbers obtained from the production software. To obtain the ablation time, an average repetition rate of 10 Hz (half of the maximum repetition rate) was used. As can be seen, the theoretical estimates match the actual numbers quite well. For myopia, the actual treatment sequence is not much different than a concentric set of pulses plus some smaller pulses to fill up the gaps.

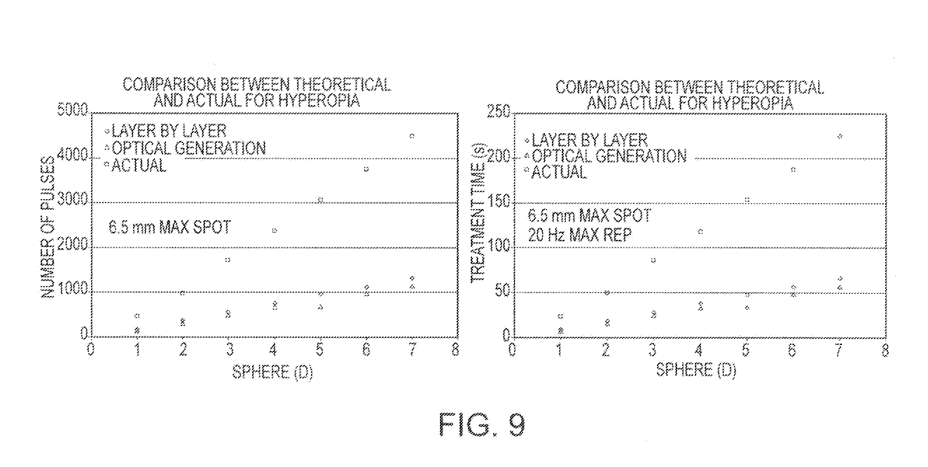

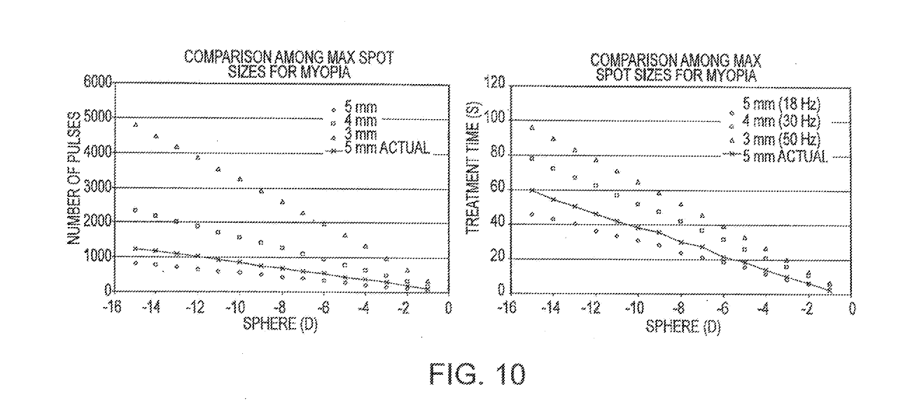

FIG. 9 provides a comparison of the number of pulses (left panel) and treatment time (right panel) between two techniques (layer by layer; optical generation) using annular shapes and the actual numbers using circular spots for hyperopia. Specifically, FIG. 9 shows the number of pulses and treatment time between the theoretically estimated numbers using annular shapes and the actual numbers using circular spots only. As can be seen, a time reduction factor of 3.1 (layer by layer basis), on average, can be achieved with the use of annular shapes as compared to only using circular shapes for hyperopia. If the energy is preserved (optical generation), the time reduction factor can be as high as 3.7. As an example, for a 7(D) treatment, the number of pulses corresponding to an actual treatment is about 4500, whereas the number of pulses corresponding to the layer by layer and optical generation treatments is about 1100 to 1200. Relatedly, for a 7(D) treatment, the treatment time corresponding to an actual treatment is about 225 seconds, whereas the treatment time corresponding to the layer by layer and optical generation treatments is about 55 to 65 seconds. Hence, annular shapes can provide improved efficiency for hyperopia treatments.

FIG. 10 provides a comparison of the number of pulses (left panel) and treatment time (right panel) for various maximum spot sizes of 5 mm, 4 mm, and 3 mm for myopia. These examples involve circular spots delivered at a 25 Hz repetition rate (maximum 50 Hz). For a higher repetition rate laser, theoretical limits for maximum spot sizes of 5 mm, 4 mm, and 3 mm were calculated, together with the actual numbers for a 5 mm higher repetition rate system. For myopia, the ablation efficiency is observed to decrease as the maximum spot size decreases. Due to the corneal temperature constraint, an average repetition rate of 18 Hz, 30 Hz, and 50 Hz was used for 5 mm, 4 mm, and 3 mm maximum spot sizes, respectively. On average, the treatment time is 85%, 150%, and 185% of the corresponding time in a higher repetition rate laser for 5 mm, 4 mm, and 3 mm maximum spot sizes, respectively. As shown here, a 5 mm maximum spot size provides a desirable ablation time for a fast laser.

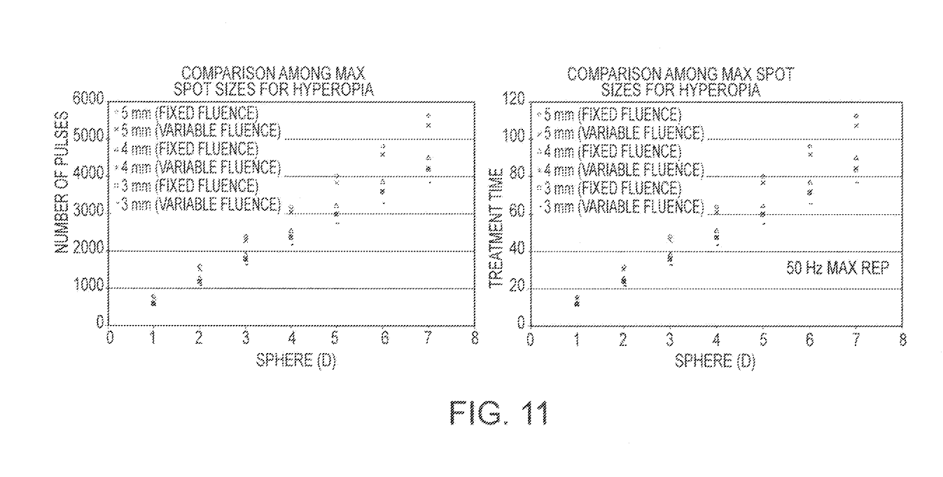

For hyperopia, the situation may be different. FIG. 11 provide a comparison of the number of pulses (left panel) and treatment time (right panel) for various maximum spot sizes of 5 mm, 4 mm, and 3 mm for hyperopia. A combination of annular and circular spots is used with 50 Hz maximum repetition rate. When the maximum spot size decreases, the ablation efficiency may actually increase slightly with the use of a combination of circular and annular basis techniques. On average, the treatment time is 150%, 120%, and 110% of the corresponding time in the actual ablation time when annular basis is used, for 5 mm, 4 mm, and 3 mm maximum spot sizes, respectively. FIG. 11 also shows aspects of fixed and variable fluence, as further described elsewhere herein.

Hence, it can be seen that for STAR, the use of a combination of annular and circular basis may not change the ablation characteristics for myopia but can increase the ablation efficiency more than three (3) times for hyperopia, all else being equal. For a higher repetition rate laser or faster broad beam laser, a decrease in maximum spot size can reduce the ablation efficiency for myopia dramatically and increase the ablation efficiency for hyperopia slightly. With a maximum repetition rate of 50 Hz, use of a 5 mm maximum spot size can reduce the average ablation time from a STAR theoretical level to about half for both myopia and hyperopia. With the use of a 4 mm maximum spot size, myopia becomes 50% longer and hyperopia 20% longer than the actual level for a faster laser. For a 3 mm maximum spot size, myopia is 85% longer and hyperopia about 10% longer than the actual level for the higher repetition rate laser, when keeping a 50 Hz maximum repetition rate. From an ablation efficiency viewpoint, a 5 mm maximum spot size appears to be a desirable.

Annular Basis Data

Annular basis data techniques described herein are well suited for use in broad beam refractive laser systems, and can provide improved treatment times and solution quality, often simultaneously. According to some embodiments, an obscuration ratio can be used when calculating and delivering annular beams. The obscuration ratio of an annular shape can refer to the ratio of the inner radius to the outer radius. Optionally, an obscuration ratio can be determined by using a different number of a set of random refractions (e.g. 5 to 10), for example through a Monte Carlo simulation. In some cases, a larger obscuration ratio can enhance solution quality. In some cases, a smaller obscuration ratio can enhance ablation efficiency. In some instances, a threshold based on iris dimensions can be used to determine whether to use annular spots or circular spots. For example, some embodiments may involve using annular spots for larger iris sizes and circular spots for smaller iris sizes. In some instances, maximum optical zone OZ and maximum iris size may be highly related.

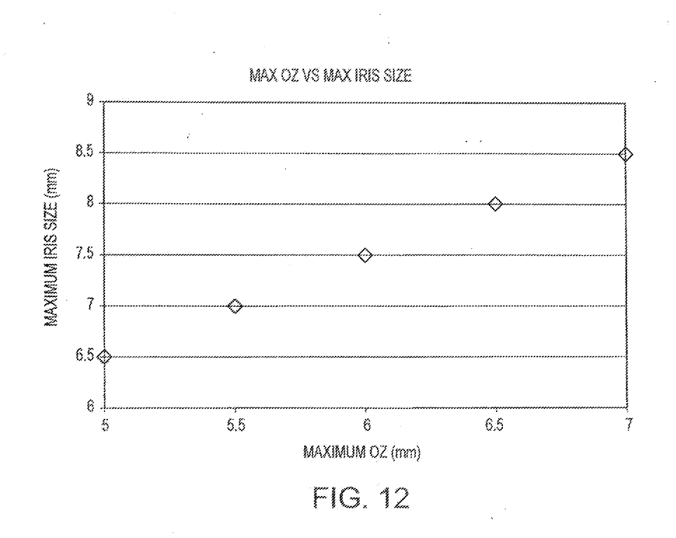

FIG. 12 provides a graphic illustration of the relationship between maximum iris size and maximum OZ, for example as processed through a Monte Carlo simulation, according to embodiments of the present invention.

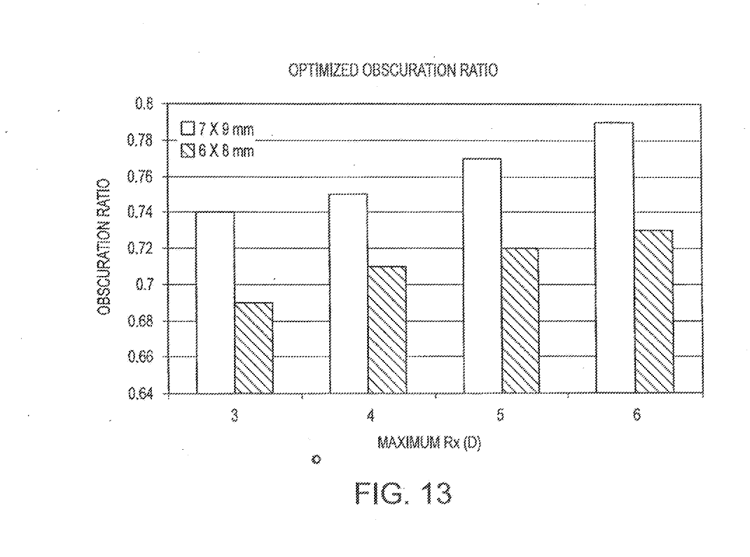

FIG. 13 provides a graphic illustration of the relationship between obscuration ratio and maximum Rx (D), for 7 mm (optical zone) ×9 mm (ablation zone) and 6 mm (optical zone) ×8 mm (ablation zone), for example as processed through a Monte Carlo simulation, according to embodiments of the present invention. The term Rx (D) can be defined as a refraction to be corrected or treated. As depicted in FIG. 13, there may be a correlation between the obscuration ratio and the maximum Rx (D). For example, as shown here, an increase in obscuration ratio corresponds to an increase in maximum Rx (D). According to an empirical evaluation, a desired obscuration ration can be about 0.75.

Experiments using different inner and outer radius values can be performed to obtain resulting PV and RMS for treatment table creation. For each Rx, a variety of combinations can be performed. It may be easier to fit a target shape where Rx values are lower, so lower obscuration ratio annular pulses can be used thus increasing the ablation efficiency. Where higher obscuration ratios are used, there may be a reduction in ablation efficiency, because thinner rings or lower volumes are being ablated.

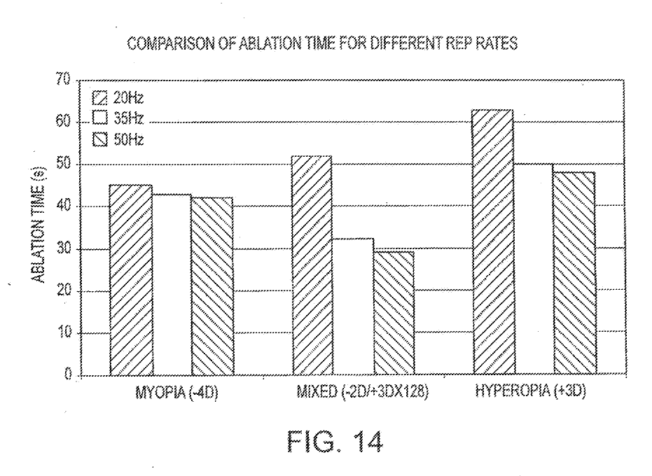

FIG. 14 provides a graphic illustration of the relationship between ablation time and myopia, mixed, and hyperopia treatments, for 20 Hz, 35 Hz, and 50 Hz repetition rates, for example as evaluated through a Monte Carlo simulation, according to embodiments of the present invention.

FIG. 15 shows the relationship between the ablation depth and distance from the iris center, for a proposed annular tissue basis data (no iris extension) corresponding to a 6.5 mm maximum iris, according to embodiments of the present invention. FIG. 15 pertains to annular ablation shapes or profiles, in comparison with the circular ablation shapes or profiled discussed with reference to FIG. 6. For example, FIG. 15 provides annular basis data for larger spots, and circular basis data for smaller spots. The break point or threshold can be, for example, between about 3 mm and about 2.5 mm. Hence, ablation pulses corresponding to an iris size that is about 3 mm or larger can be annular, and ablation pulses corresponding to an iris size that is about 2.5 mm or smaller can be circular. In some cases, the break point can be a predetermined or fixed value. In some cases, the break point can be an adjustable or free variable.

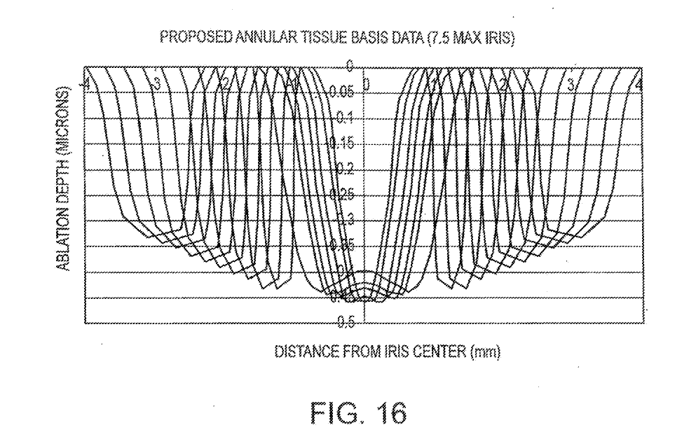

FIG. 16 shows the relationship between ablation depth and distance from iris center, for a proposed annular tissue basis data corresponding to a 7.5 mm maximum iris, according to embodiments of the present invention. In some cases, the iris may be extended from a 6.5 mm maximum to a 7.5 mm maximum.

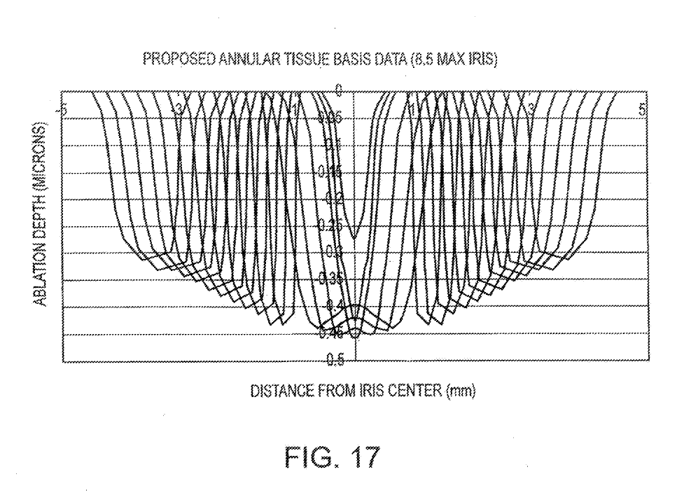

FIG. 17 shows the relationship between ablation depth and distance from iris center, for a proposed annular tissue basis data corresponding to an 8.5 mm maximum iris, according to embodiments of the present invention. In some cases, the iris may be extended from a 6.5 mm maximum to a 8.5 mm maximum. Some current basis data implementations involve a maximum iris size of 8.0 mm, and can be modified for use with a maximum iris size of 8.5 mm or more, for example 10.0 mm which corresponds to a maximum ablation area.

In some cases, an annular basis can involve annular outer radius values of 6.5 mm, 6 mm, 5.5 mm, 5 mm, 4.5 mm, 4 mm, and 3.5 mm and annular inner radius values of 4.875 mm, 4.5 mm, 4.125 mm, 3.75 mm, 3.375 mm, 3 mm, and 2.625 mm. Optionally, embodiments may involve circular radius values of 3 mm, 2.5 mm, 2 mm, 1.5 mm, and 1.0 mm. In some instances, the inner radius can be realized by high absorption material or optical means. A treatment table may be presorted to reduce mechanical rotation, and thermal aspects may be considered, optionally at the same time.

Mechanical Block (MB)

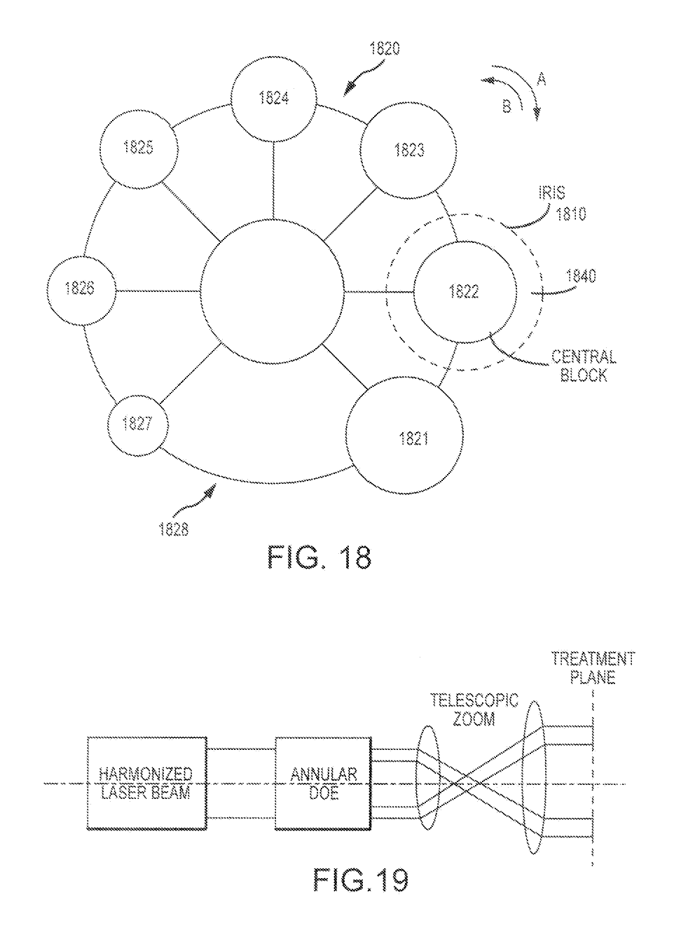

FIG. 18 relates to a mechanical block mechanism or technique for creating an annular ablation pulse shape. Table 1 shows exemplary mechanical block parameters according to embodiments of the present invention.

TABLE 1

| | | |
|---|---|---|
| Maximum Iris | Mechanical Block | |
| Dimension | Dimension | Obscuration Ratio |
| (outer diameter OD) | (inner diameter ID) | ID:OD |

| | | | |
|---|---|---|---|
| 6.5 mm (annular) | 4.875 | mm | 4.875:6.5 = 0.75 |
| 6.0 mm (annular) | 4.5 | mm | 4.5:6.0 = 0.75 |
| 5.5 mm (annular) | 4.125 | mm | 4.125:5.5 = 0.75 |
| 5.0 mm (annular) | 3.75 | mm | 3.75:5.0 = 0.75 |
| 4.5 mm (annular) | 3.375 | mm | 3.375:4.5 = 0.75 |
| 4.0 mm (annular) | 3.0 | mm | 3.0:4.0 = 0.75 |
| 3.5 mm (annular) | 2.625 | mm | 2.625:3.5 = 0.75 |
| 3.0 mm (circular) | 0 | mm | 0 |
| 2.5 mm (circular) | 0 | mm | 0 |
| 1.5 mm (circular) | 0 | mm | 0 |
| 1.0 mm (circular) | 0 | mm | 0 |

In comparison to a DOE technique, which may achieve an annular ablation pulse shape by redistributing energy in a way that conserves all or substantially all of the energy, there is typically a greater energy loss when using an MB technique when transforming a circular shape to an annular shape, because the blocked or obscured energy is not conserved.

FIG. 18 shows aspects of a mechanical block assembly 1800 according to embodiments of the present invention. Mechanical block assembly 1800 includes an adjustable iris mechanism 1810, represented by the dashed line, and a central block mechanism 1820 that can rotate about an axis 1830 in a clockwise direction as indicated by arrow A or a counterclockwise direction as indicated by arrow B. Central block mechanism 1820 can include one or more obscuration elements. For example, central block mechanism 1820 can include a first obscuration element 1821, a second obscuration element 1822, a third obscuration element 1823, a fourth obscuration element 1824, a fifth obscuration element 1825, a sixth obscuration element 1826, and a seventh obscuration element 1827. Central block mechanism 1820 can be rotatably adjusted such that an obscuration element is positioned along the path of the laser beam and aligned with iris mechanism 1810. As shown here, second obscuration element 1822 is positioned relative to iris mechanism 1810, such that an annular portion of the laser beam is transmitted through the annular shaped passage 1840 while the central portion of the laser beam is blocked by obscuration element 1822. Iris mechanism 1810 can be adjusted to any dimension as desired. For example, with reference to Table 1, iris mechanism 1810 can be adjusted to 6.5 mm outer diameter, a 6.0 mm outer diameter, a 5.5 mm outer diameter, and the like. Relatedly, central block mechanism 1820 can provide a 4.875 mm obscuration block, a 4.5 mm obscuration block, a 4.125 obscuration block, and the like, for the inner diameter dimension. In some cases where a circular ablation pulse or shape is desired, central block mechanisms can be adjusted, for example so that an obscuration blank 1828 is aligned with iris 1810, so that a laser beam can pass through iris 1810 without obscuration of a central portion of the laser beam. In some cases, central block mechanism may be rotated by a motor mechanism.

It is possible to pre-sort the laser ablation pulses for hyperopia in such a way that pulses with the same central obscuration blocks are ablated in the same sequence. Such pre-sorting can reduce the amount of obscuration block changes or switches. The use of mechanical block assembly 1800 provides an optical approach to reshape the laser beam a such a way that it can modify either the central block size or both the inner and outer diameter of the entire aperture. Also, embodiments encompass optical ways to image, redistribute energy, and extend beyond current largest aperture. Hardware variations provide for the production of an annular shape using a mechanical approach or an optical approach. Exemplary techniques may include a double spot by bifringent protocol, for example which uses a double spot shape as shown in FIG. 5, or an elliptical and arc by beam reshaping protocol, for example which uses a double crescent spot shape as shown in FIG. 5. In some embodiments, administration of a hyperopia treatment involves adjusting central block mechanism 1820 so that a combination of various obscuration elements, and optionally the obscuration blank, are sequentially aligned with the iris during transmission of the laser pulses. In some embodiments, administration of a myopia treatment involves setting the central block mechanism so that the obscuration blank is aligned with the iris throughout transmission of the laser pulses.

Diffractive Optical Element (DOE)

According to some embodiments, it is possible to use lithographically ruled gratings with diffractive optics to redistribute energy into annular shapes. In some cases, using a mechanical switch to alternate circular and annular pulses, pulses can be presorted so only one switch is involved. Laser voltage can be controlled such that the fluence on a treatment plane is constant. In some instances, a rotational symmetry is built-in to a SALSA algorithm so an algorithm change may be involved. Optionally, a DOE approach may involve instantaneous switching between circular and annular spot shapes during administration of a treatment.

As mentioned elsewhere herein, FIG. 11 shows aspects of fixed and variable fluence. For DOE, annular ablation shapes can be generated in various ways. For example, one way may involve a natural technique that includes directing all output energy into the annular area, which makes the fluence variable. Another way may involve directing partial output energy into the annular area such that the fluence (energy per unit area) is fixed, which can be used in conjunction with current basis data architecture for the implementation of the annular pulses.

FIG. 19 shows a simplified optical set up, according to embodiments of the present invention. In some cases, a proposed annular ratio (inner/outer) can be 0.75.

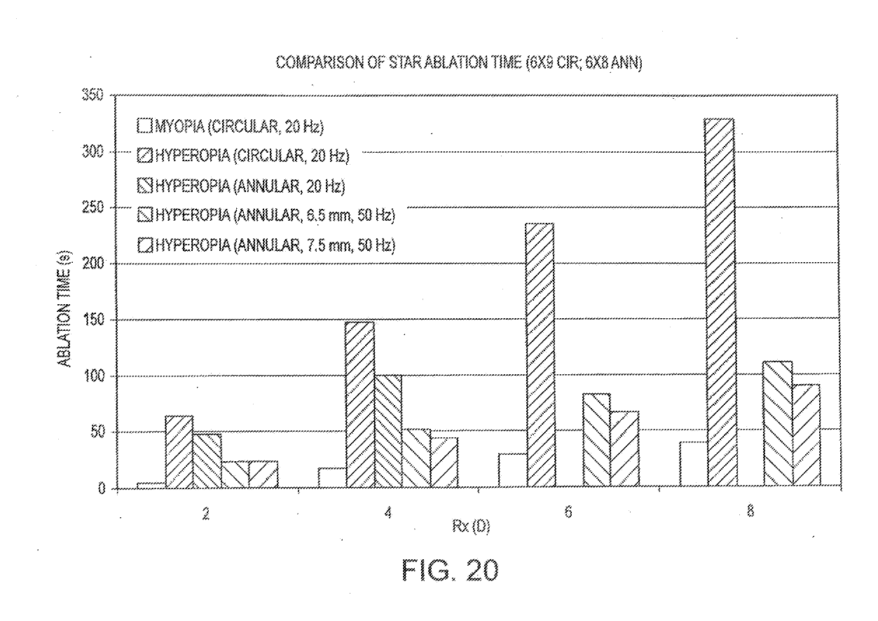

FIG. 20 shows a comparison of STAR ablation times for 6 mm (optical zone) ×9 mm (ablation zone) circular and 6 mm (optical zone) ×8 mm (ablation zone) annular embodiments, in myopia and hyperopia treatments. These treatment time estimates are provided for a 6 mm OZ.

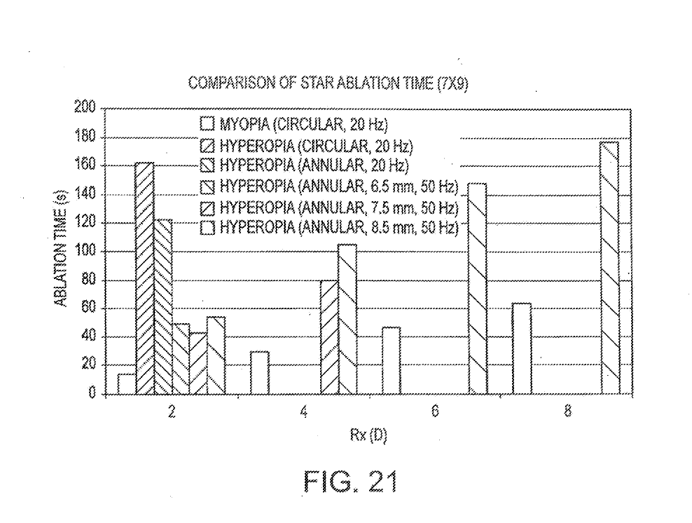

FIG. 21 shows a comparison of STAR ablation times for 7 mm (optical zone) ×9 mm (ablation zone) embodiments, for different basis data implementations, in myopia and hyperopia treatments. These treatment time estimates are provided for a 7 mm OZ.

As illustrated in FIGS. 20 and 21, when the optical zone OZ changes for purposes of hyperopia, the optional maximum iris size can also change. For example, for a 5 mm OZ, a 6.5 mm maximum iris size can be used. Relatedly, for a 6 mm OZ, a 7.5 mm maximum iris size can be used. Further, for an 8 mm OZ, a 9.5 maximum iris size can be used. Hence, embodiments encompass the implementation of optimal maximum iris sizes for different optical zone settings.

Embodiments of the present invention encompass various approaches for revising or generating basis data. In some cases, simulations and other techniques can be used to determine desirable annular ratios as well as the separation of annular and circular pulse sizes. Bench work can be performed using calibration plastics for various annular shapes, for example with a STAR laser system. Verification work can be done with eye ablation for verification. Shapes may be revised based on a controlled clinical study with 10 to 20 eyes, for example. Basis data files and algorithms can be revised or developed to include annular spots.

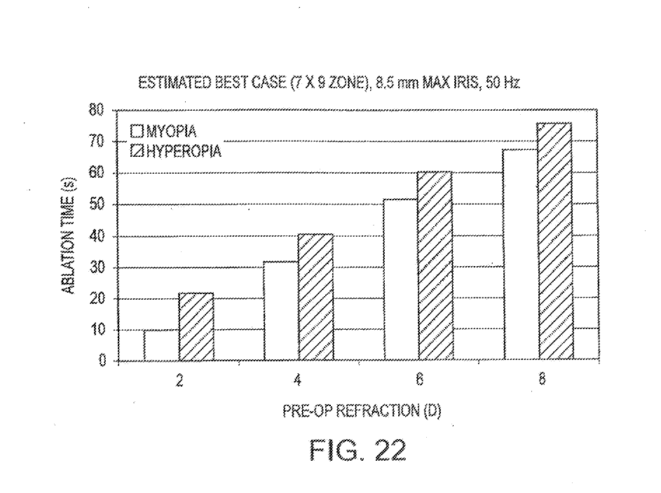

FIG. 22 shows an estimated or theoretical relationship between ablation time and pre-operative refraction, for myopia and hyperopia, corresponding to a 7 mm (optical zone) ×9 mm (ablation zone), 8.5 mm maximum iris, and 50 Hz repetition rate. Hence, it can be seen that hyperopia ablation treatments using annular pulse shapes can be administered at levels of efficiency equivalent to those achieved with myopia ablation treatments using circular shapes.

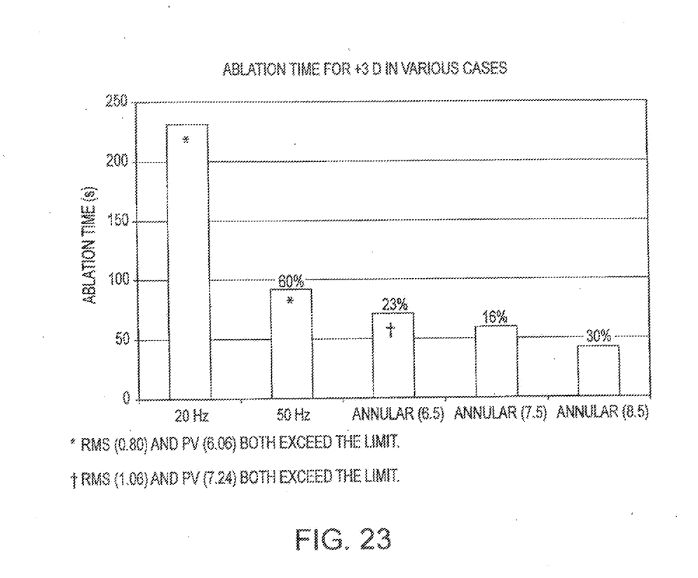

FIG. 23 shows the ablation time for a +3 D hyperopia [7 mm (optical zone) ×9 mm (ablation zone)] treatment in various cases. In the 20 Hz and 50 Hz repetition rate examples, RMS (0.80) and PV (6.06) both exceed the limit. In the annular 6.5 mm maximum iris size example, RMS (1.06) and PV (7.24) both exceed the limits of solution quality criteria or tolerances. These limits can be expressed as RMS and PV between a theoretical target shape and an ablated shape.

Implementation of annular shapes for vision treatment can be effected in a variety of ways. In some cases, particular implementations can be based on an iris size limit. For example, in some cases involving a 6.5 mm iris size limit, embodiments may encompass using a mechanical block technique, which can provide a 20% increase in speed without optimization, and a 50% increase with 50 Hz. In some cases involving an extended 7.5 mm iris size limit, embodiments may encompass using mechanical block and diffractive optical element techniques, which can provide a 200% increase in speed even with 20 Hz. In some cases involving an extended 8.5 mm iris size limit, embodiments may include hyperopia treatment speeds which are similar to similar to myopia treatment speeds, at any repetition rate.

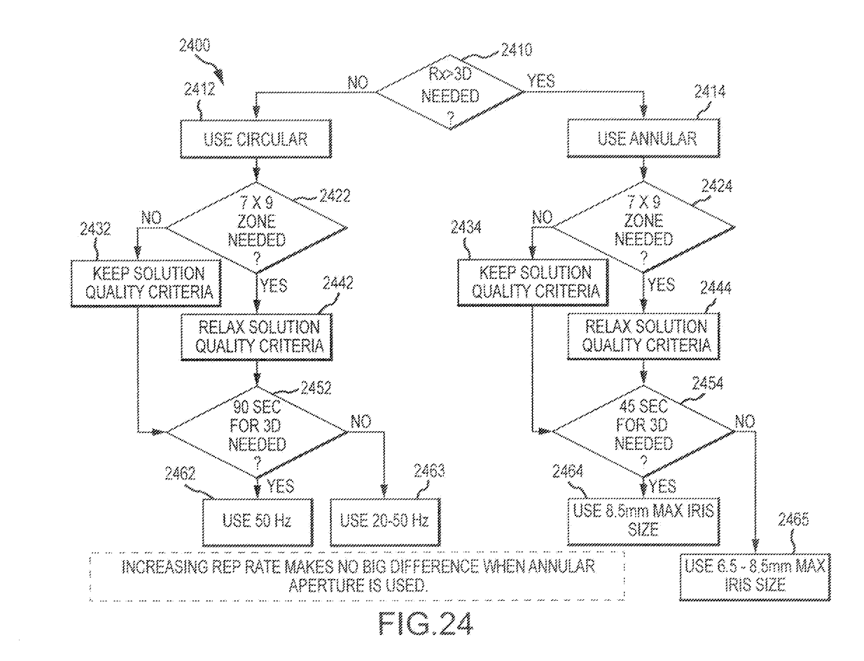

FIG. 24 provides a decision tree or method 2400 associated with an intended approval range for refractive treatments, or with approaches for building a treatment system, according to embodiments of the present invention. Step 2410 of the decision tree or method involves determining whether an Rx of greater than a particular treatment threshold, for example 3D, is desired. Other threshold values may be used. If the Rx desired or needed is not greater than the treatment threshold, then the method involves using a circular ablation or pulse shape, as indicated by step 2412. If the Rx desired or needed is greater than the treatment threshold, then the method involves using an annular ablation or pulse shape, as indicated by step 2414.

According to some embodiments, the left side of the decision tree pertains to a lower or normal hyperopia treatment, for example less than about 3D, and the right side pertains to a higher hyperopia treatment, for example more than about 3D. The left side can also pertain to a myopia treatment. Relatedly, the left side involves administration of circular ablation pulses, often exclusively, whereas the right side involves administration of annular ablation pulses, optionally in combination with circular pulses. The break point or threshold, for example 3D, can have implications for ablation depth, treatment time, and solution quality. In the event that a circular ablation or pulse shape is used, step 2422 can be performed to determine whether a 7 mm (optical zone) ×9 mm (ablation zone) is desired. Embodiments may encompass the use of other optical zone OZ and ablation zone AZ values. In the event that an annular ablation or pulse shape is used, step 2424 can be performed to determine whether a 7 mm (optical zone) ×9 mm (ablation zone) is desired.

Where a circular ablation or pulse shape is used, and a 7 mm (optical zone) ×9 mm (ablation zone) is not used, a decision can be made to keep the solution quality criteria, as indicated by step 2432. Where a circular ablation or pulse shape is used, and a 7 mm (optical zone) ×9 mm (ablation zone) is used, a decision can be made to relax the solution quality criteria, as indicated by step 2442. Where an annular ablation or pulse shape is used, and a 7 mm (optical zone) ×9 mm (ablation zone) is not used, a decision can be made to keep the solution quality criteria, as indicated by step 2434. Where an annular ablation or pulse shape is used, and a 7 mm (optical zone) ×9 mm (ablation zone) is used, a decision can be made to relax the solution quality criteria, as indicated by step 2444.

Where a circular ablation or pulse shape is used, the method can involve determining whether a particular time threshold for the treatment threshold is desired or needed, as indicated by step 2452. For example, the method step may include determining whether a 90 second treatment time for a 3D treatment is desired or needed. As shown here, if a 90 second treatment time for a 3D treatment is desired or needed, then a 50 Hz repetition rate can be used, as indicated by step 2462. If a 90 second treatment time for a 3D treatment is not desired or needed, then a 20-50 Hz repetition rate can be used, as indicated by step 2463. Where an annular ablation or pulse shape is used, the method can involve determining whether a particular time threshold for the treatment threshold is desired or needed, as indicated by step 2454. For example, the method step may include determining whether a 45 second treatment time for a 3D treatment is desired or needed. As shown here, if a 45 second treatment time for a 3D treatment is desired or needed, then an 8.5 mm maximum iris size can be used, as indicated by step 2464. Such approaches can be implemented when a faster treatment is desired. If a 45 second treatment time for a 3D treatment is not desired or needed, then a 6.5-8.5 mm maximum iris size can be used, as indicated by step 2465. Such approaches can be implemented when a slower treatment is suitable. As indicated in FIG. 24, when an annular aperture is being used, which covers a greater amount of surface area and thus confers enhanced efficiency, an increase in repetition rate will typically not affect the outcome. In contrast, when using circular spots, the ablation time can be longer, and hence repetition rate can play a more significant role. Faster ablation procedures can help to avoid complications related to dehydration.

The methods and apparatuses of the present invention may be provided in one or more kits for such use. The kits may comprise a system for profiling an optical surface, such as an optical surface of an eye, and instructions for use. Optionally, such kits may further include any of the other system components described in relation to the present invention and any other materials or items relevant to the present invention. The instructions for use can set forth any of the methods as described herein.

Each of the calculations or operations described herein may be performed using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described above. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like.

All patents, patent publications, patent applications, journal articles, books, technical references, and the like discussed in the instant disclosure are incorporated herein by reference in their entirety for all purposes.

While the above provides a full and complete disclosure of the preferred embodiments of the present invention, various modifications, alternate constructions and equivalents may be employed as desired. Therefore, the above description and illustrations should not be construed as limiting the invention, which can be defined by the claims.

What is claimed is:

1. A system for ablating optical tissue in an eye of a patient, the system comprising:
a laser mechanism that generates multiple laser beam pulses, each laser beam pulse having an original geometry;
a mechanical block mechanism that transforms the laser beam pulses, each transformed laser beam pulse having an annular geometry, the mechanical block mechanism having an adjustable iris mechanism and a central block mechanism including one or more obscuration elements that can be positioned along a laser beam path and aligned with the adjustable iris mechanism to define an annular shaped passage; and
a delivery mechanism that directs the transformed laser beam pulses toward the eye of the patient so as to ablate the optical tissue,
wherein the obscuration element is positionable to block an inner portion of the original geometry laser beam pulse, and an outer portion of the original geometry laser beam pulse transmitted through the annular shaped passage provides the transformed laser beam pulse having the annular geometry.

2. The system according to claim 1, wherein the central block mechanism is configured to rotate about an axis, such that the one or more obscuration elements are independently positionable relative to the adjustable iris mechanism to define the annular shaped passage.

3. The system according to claim 1, wherein the central block mechanism comprises an obscuration blank positionable relative to the adjustable iris mechanism, such that the adjustable iris mechanism in combination with the obscuration blank provides a circular shaped passage for transmission of the laser beam pulses.

4. The system according to claim 1, wherein adjustable iris mechanism is adjustable to an outer diameter selected from the group consisting of 1.0 mm, 1.5 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, and 10.0 mm.

5. The system according to claim 1, wherein the adjustable iris mechanism is adjustable to an outer diameter within a range from about 1.0 mm to about 10.0 mm.

6. The system according to claim 1, wherein the one or more obscuration elements present a mechanical block inner diameter within a range from about 2.625 mm to about 7.5 mm.

7. The system according to claim 1, wherein the adjustable iris mechanism defines a mechanical block outer diameter and the one or more obscuration elements define a mechanical block inner diameter, such that an obscuration ratio calculated by dividing the inner diameter by the outer diameter is about 0.75.

8. A method for ablating optical tissue in an eye of a patient, the method comprising:
generating multiple laser beam pulses with a laser, each laser beam pulse having an original geometry;
transforming the laser beam pulses with a mechanical block mechanism, each transformed laser beam pulse having an annular geometry; and
directing the transformed laser beam pulses toward the eye of the patient so as to ablate the optical tissue.

9. The method according to claim 8, wherein the directing step comprises scanning the transformed laser beam pulses toward the eye in a nondeterministic pattern.

10. The method according to claim 8, wherein the annular geometry is determined based on an optimal obscuration ratio.

11. The method according to claim 8, wherein the transforming step is performed based on an outer dimension of the laser beam pulse.

12. The method according to claim 8, wherein the annular geometry comprises an outer dimension that is determined based on an optical zone of the patient.

13. A system for ablating optical tissue in an eye of a patient, the system comprising:
a laser mechanism that generates multiple laser beam pulses, each laser beam pulse having an original geometry;
a mechanical block mechanism that transforms the laser beam pulses, each transformed laser beam pulse having an annular geometry; and
a delivery mechanism that directs the transformed laser beam pulses toward the eye of the patient so as to ablate the optical tissue.

14. The system according to claim 13, wherein the delivery mechanism is configured to scan the transformed laser beam pulses toward the eye in a nondeterministic pattern.

15. The system according to claim 13, wherein the mechanical block mechanism is configured to transform the laser beam pulses to the annular geometry based on an optimal obscuration ratio.

16. The system according to claim 13, wherein the mechanical block mechanism is configured to transform the laser beam pulses to the annular geometry based on an outer dimension of the laser beam pulse.

17. The system according to claim 13, wherein the mechanical block mechanism is configured to transform the laser beam pulses to the annular geometry having an outer dimension that is determined based on an optical zone of the patient.

18. The system according to claim 13, wherein the delivery mechanism is configured to direct the transformed laser beam pulses toward the eye of the patient in a nonconcentric pattern.

19. The system according to claim 13, wherein the mechanical block mechanism comprises an adjustable iris mechanism and a central block mechanism including one or more obscuration elements that can be positioned along a laser beam path and aligned with the adjustable iris mechanism to define an annular shaped passage.

20. The system according to claim 19, wherein the one or more obscuration elements are independently positionable relative to the adjustable iris mechanism to block an inner portion of the original geometry laser beam pulse, and an outer portion of the original geometry laser beam pulse transmitted through the annular shaped passage provides the transformed laser beam pulse having the annular geometry.
